(19) United States
(12) Patent Application Publication
(10) Pub. No.: US 2025/0304917 A1
(43) Pub. Date: Oct. 2, 2025

(54) A METHOD FOR SEPARATING ADENO-ASSOCIATED VIRUS CAPSIDS, COMPOSITIONS OBTAINED BY SAID METHOD AND USES THEREOF

(71) Applicant: Cytiva BioProcess R&D AB, Uppsala (SE)

(72) Inventors: Jean-Luc Maloisel, Uppsala (SE); Asa Hagner McWhirter, Uppsala (SE); Ola Lind, Uppsala (SE); Hans Blom, Uppsala (SE)

(21) Appl. No.: 18/578,251

(22) PCT Filed: Apr. 27, 2022
(86) PCT No.: PCT/EP2022/061257

§ 371 Date: Jan. 10, 2024

(30) Foreign Application Priority Data
Jul. 12, 2021 (GB) 2110014.4

(51) Int. Cl. C12N 7/00; A61K 48/00; B01J 41/20; B01J 47/02; C12N 15/86

(52) U.S. Cl. CPC C12N 7/00; A61K 48/0041; B01J 41/20; B01J 47/02; C12N 15/86; C12N 2750/14143; C12N 2750/14151

(57) ABSTRACT

The present disclosure is directed to a method for separating adeno-associated virus capsids fully packaged with genetic material from adeno-associated virus capsids not fully packaged with genetic material, the method comprising the following steps: a) adding a liquid sample comprising adeno-associated virus capsids to a chromatography material, wherein the liquid sample comprises adeno-associated virus capsids of a purity of at least 90% and of a concentration of at least 1012 adeno-associated virus capsids/ml, of which at least 10% of the adeno-associated virus capsids are adeno-associated virus capsids fully packaged with genetic material, wherein the chromatography material comprises a strong, or partially strong, anion exchange chromatography material comprising a support and a ligand for binding to the adeno-associated virus capsids; wherein the chromatography material comprises a surface extender connecting the ligand to the support, wherein the surface extender is a polymer, wherein the polymer is selected from: (i) a polymer having a naturally occurring skeleton, such as a polysaccharide, such as starch, cellulose, dextran, or agarose; and (ii) a polymer having a synthetic skeleton, such as a polyvinyl alcohol, a polyacrylamide, a polymethacrylamide, or a polyvinyl ether; b) eluting the adeno-associated virus capsids fully packaged with genetic material from the chromatography material; wherein the adeno-associated virus capsids eluted in step (b) are eluted into eluate fractions, which eluate fractions combined comprise at least 50% of the adeno-associated virus capsids of the liquid sample added in step (a), of which at least 60% of the adeno-associated virus capsids are fully packaged with genetic material. Further disclosed are compositions, including pharmaceutical compositions, obtained by said separation method, as well as uses of such compositions, and uses of an anion chromatography material for separation of adeno-associated virus capsids.

### FIELD OF THE DISCLOSURE

The present disclosure relates to the field of separation of adeno-associated capsids and is directed to a method for separating adeno-associated virus capsids fully packaged with genetic material from adeno-associated virus capsids not fully packaged with genetic material, and use of an anion exchange chromatography material for such separations. Further disclosed are compositions, including pharmaceutical compositions, obtained by said method as well as uses of such compositions.

### BACKGROUND OF THE DISCLOSURE

Adeno-associated viruses (AAV) are non-enveloped viruses that have linear single-stranded DNA (ssDNA) genome and that can be engineered to deliver DNA to target cells. Recombinant adeno-associated virus (rAAV) vectors have emerged as one of the most versatile and successful gene therapy delivery vehicles. There is an increasing demand to use viral vectors for gene therapy. The AAV vector is one of the most attractive gene transfer tools for developing novel genetic therapies for muscle diseases as well as other disorders. Most of the earlier AAV gene transfer studies used AAV serotype 2 (AAV2). To further improve the efficiency and specificity of AAV-mediated gene transfer, numerous AAV serotypes and variants have been developed by viral genome engineering and/or capsid modification. Use of serotypes like AAV8 and AAV9 have increased in recent years. Target organs determine selection of serotype. To use AAV particles as vectors in therapy it is necessary to purify the virus particles from cell impurities like DNA after transfection. Ultracentrifugation is efficient but not scalable. Normally, several filtration steps and several chromatography steps are used to separate AAV particles from cell cultures (see e.g., Weihong Qu et al, Scalable Downstream Strategies for Purification of Recombinant Adeno-Associated Virus Vectors in Light of the Properties, Current Pharmaceutical Biotechnology 2015 Aug.; 16(8): 684-695).

Therapeutic efficacy of AAV vectors is dependent on high percentage of virus particles fully packaged with genetic material of interest. Upstream expression systems deliver a mixture of fully packaged AAV particles (containing the genetic material of interest), empty AAV particles, and AAV particles which are partially packaged with genetic material of interest), together with impurities. There is thus a need to enrich fully packaged AAV particles in the purification process. However, there are several challenges in relation to achieving an efficient and scalable separation of fully packaged and empty adeno-associated virus capsids, such as:
Large diversity of capsids (serotypes and variants) and cell culture differences in terms of yield of full capsids, which means that extensive optimization is needed for purification of each serotype or variant of adeno-associated virus.
Small differences between fully packaged and empty capsids in relation to several parameters relevant for purification, e.g. isoelectric point;
In addition to fully packaged and empty capsids, also partially packaged capsid variants are produced in the infected host cells. There are indications that such partially packaged, and thereby therapeutically less effective, capsids may be partly co-eluted with fully packaged capsids.

Consequently, novel purification strategies are required to increase the speed and decrease the cost of the purification process and to provide large-scale methods for downstream processing of different adeno-associated virus serotypes.

### SUMMARY OF THE DISCLOSURE

The object of the present disclosure is to provide a scalable solution providing an improved separation of fully packaged adeno-associated virus capsids from not fully packaged adeno-associated virus capsids. This is achieved by obtaining an improved resolution between fully packaged and not fully packaged capsids when performing a separation method as disclosed herein, which results in achieving a composition having an improved ratio of fully packaged capsids to not fully packaged capsids. The focus of the disclosure is the polishing step of a separation method, also called secondary or final purification.

More particularly, a first aspect of the present disclosure is directed to a method for separating adeno-associated virus capsids fully packaged with genetic material from adeno-associated virus capsids not fully packaged with genetic material, the method comprising the following steps:

a) adding a liquid sample comprising adeno-associated virus capsids to a chromatography material, wherein the liquid sample comprises adeno-associated virus capsids of a purity of at least 90% and of a concentration of at least 10¹² adeno-associated virus capsids/ml, of which at least 10% of the adeno-associated virus capsids are adeno-associated virus capsids fully packaged with genetic material,
wherein the chromatography material comprises a strong, or partially strong, anion exchange chromatography material comprising a support and a ligand for binding to the adeno-associated virus capsids;
wherein the chromatography material comprises a surface extender connecting the ligand to the support, wherein the surface extender is a polymer, wherein the polymer is selected from:
(i) a polymer having a naturally occurring skeleton, such as a polysaccharide, such as starch, cellulose, dextran, or agarose; and
(ii) a polymer having a synthetic skeleton, such as a polyvinyl alcohol, a polyacrylamide, a polymethacrylamide, or a polyvinyl ether;
   b) eluting the adeno-associated virus capsids fully packaged with genetic material from the chromatography material;
   wherein the adeno-associated virus capsids eluted in step (b) are eluted into eluate fractions, which eluate fractions combined comprise at least 50% of the adeno-associated virus capsids of the liquid sample added in step (a), of which at least 60% of the adeno-associated virus capsids are fully packaged with genetic material.

The above-disclosed method may further comprise subjecting the eluate fractions comprising adeno-associated virus capsids fully packaged with genetic material, eluted in step (b), to one or more of the following steps:

c1) concentrating the adeno-associated virus capsids to a pharmaceutically relevant dose,
c2) replacing a buffer applied in step (b) of the above-mentioned separation method with a pharmaceutically acceptable buffer, and/or
c3) sterilizing the eluate fractions comprising adeno-associated virus capsids, thereby obtaining a pharmaceutical composition comprising adeno-associated virus capsids.

The present disclosure further provides a method for preventing or treating a disease or disorder related to an organ or tissue in a subject, comprising administering to the subject a pharmaceutical composition comprising adeno-associated virus capsids obtained by performing the above-mentioned separation method, in which pharmaceutical composition the ratio of adeno-associated virus capsids fully packaged with genetic material to adeno-associated virus capsids not fully packaged with genetic material is at least 3:2.

Additionally, the present disclosure is directed to a composition comprising adeno-associated virus capsids obtained by performing the method for separating fully packaged adeno-associated virus capsids from not fully packaged adeno-associated virus capsids as described in detail above, in which composition the ratio of adeno-associated virus capsids fully packaged with genetic material to adeno-associated virus capsids not fully packaged with genetic material is at least 3:2.

The present disclosure also provides a pharmaceutical composition comprising adeno-associated virus capsids obtained by performing the above-disclosed separation method comprising one or more of steps c1-c3 (as described in detail above), in which pharmaceutical composition the ratio of adeno-associated virus capsids fully packaged with genetic material to adeno-associated virus capsids not fully packaged with genetic material is at least 3:2.

Further provided is the above-described pharmaceutical composition for use in therapy, optionally for use in gene therapy.

The present disclosure also provides use of an anion exchange chromatography material comprising a support, a ligand, and a surface extender connecting the ligand to the support, and being defined by Formula IV:

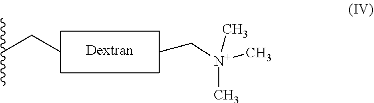

for separating adeno-associated virus capsids fully packaged with genetic material from adeno-associated virus capsids not fully packaged with genetic material, as described in detail further below.

In particular, the present disclosure is directed to separation and uses of adeno-associated virus capsids of adeno-associated virus serotypes 1, 2, 4, 5, 6, 7, 8, 9, and 10 (AAV1, AAV2, AAV4, AAV5, AAV6, AAV7, AAV8, AAV9, and AAV10) or a variant thereof.

Preferred aspects of the present disclosure are described below in the detailed description and in the dependent claims.

### BRIEF DESCRIPTION OF THE DRAWINGS

FIG. 1 is a flow chart of a method for separating adeno-associated virus capsids according to the present disclosure.

FIG. 2 is a flow chart of the method for separating adeno-associated virus capsids according to FIG. 1, further comprising an additional step (a1) before step (a).

FIG. 3 is a flow chart of the method for separating adeno-associated virus capsids according to FIG. 1, optionally comprising step (a1) before step (a), further comprising one or more additional steps (c1), (c2) and/or (c3) after step (b).

FIG. 4 is a graph showing the elution curves for fully packaged and empty capsids according to separation methods as described in Example 1 below.

FIG. 5 is a graph showing the elution curves for fully packaged and empty capsids according to a separation method as described in Example 1 below.

FIG. 6 A-D are graphs showing the elution curves for fully packaged and empty capsids according to separation methods as described in Example 4 below.

FIG. 7 is a graph showing the elution curve for fully packaged and empty capsids according to a separation method as described in Example 4 below.

FIG. 8 is a graph showing the elution curve for fully packaged and empty capsids according to a separation method as described in Example 5 below.

FIG. 9 is a graph showing the elution curve for fully packaged and empty capsids according to a separation method as described in Example 6 below.

### DETAILED DESCRIPTION OF THE DISCLOSURE

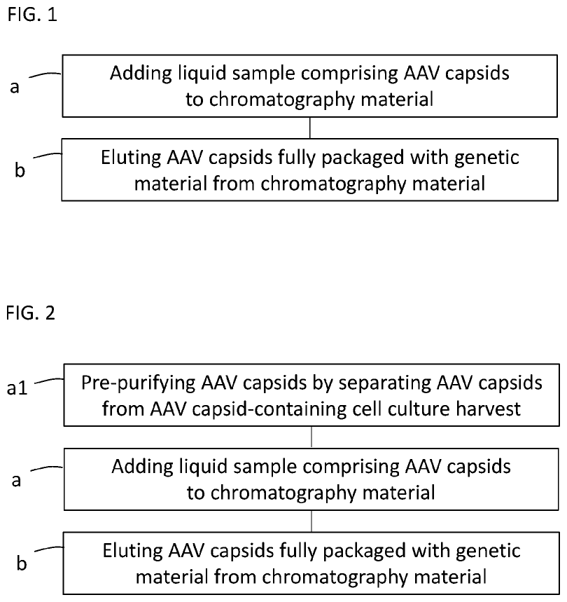

The present disclosure solves or at least mitigates the problems associated with existing methods for separating fully packaged adeno-associated virus capsids from capsids not fully packaged adeno-associated virus capsids by providing, as illustrated in FIG. 1, a method for separating adeno-associated virus capsids fully packaged with genetic material from adeno-associated virus capsids not fully packaged with genetic material, the method comprising the following steps:

a) adding a liquid sample comprising adeno-associated virus capsids to a chromatography material,
wherein the liquid sample comprises adeno-associated virus capsids of a purity of at least 90% and of a concentration of at least 10¹² adeno-associated virus capsids/ml, of which at least 10% of the adeno-associated virus capsids are adeno-associated virus capsids fully packaged with genetic material,
wherein the chromatography material comprises a strong, or partially strong, anion exchange chromatography material comprising a support and a ligand for binding to the adeno-associated virus capsids;
wherein the chromatography material comprises a surface extender connecting the ligand to the support, wherein the surface extender is a polymer, wherein the polymer is selected from:
(i) a polymer having a naturally occurring skeleton, such as a polysaccharide, such as starch, cellulose, dextran, or agarose; and
(ii) a polymer having a synthetic skeleton, such as a polyvinyl alcohol, a polyacrylamide, a polymethacrylamide, or a polyvinyl ether;
   b) eluting the adeno-associated virus capsids fully packaged with genetic material from the chromatography material;
   wherein the adeno-associated virus capsids eluted in step (b) are eluted into eluate fractions, which eluate fractions combined comprise at least 50% of the adeno-associated virus capsids of the liquid sample added in step (a), of which at least 60% of the adeno-associated virus capsids are fully packaged with genetic material.

Significant advantages of the presently disclosed method include that it is suitable for large-scale separation of fully packaged capsids from not fully packaged capsids and that it provides an improved ratio of fully packaged to not fully packaged capsids compared to prior art methods. By performing the presently disclosed method, it is possible to obtain a composition which has a ratio of fully packaged capsids to not fully packaged capsids of at least 3:2. More particularly, the presently disclosed method provides an improved ratio of fully packaged capsids to empty capsids, as well as an improved ratio of fully packaged capsids to partially packaged capsids.

A “virus particle” is herein used to denote a complete infectious virus particle. It includes a core, comprising the genome of the virus (i.e., the viral genome), either in the form of ribonucleic acid (RNA) or deoxyribonucleic acid (DNA), and the core is surrounded by a morphologically defined shell. The shell is called a capsid. The capsid and the enclosed viral genome together constitute the so-called nucleocapsid. The nucleocapsid of some viruses is surrounded by a lipoprotein bilayer envelope. In the field of bioprocessing, for the purpose of producing viral vectors for various applications such as therapy, the genome of a virus particle is modified to include a genetic insert, comprising genetic material of interest. Modified virus particles are allowed to infect host cells in a cell culture and the virus particles are propagated in said host cells, after which the virus particles are purified from the cell culture by any means of separation and purification. Herein, a virus particle to be separated from a cell culture by the presently disclosed method may alternatively be referred to as a “target molecule” or “target”. It is to be understood that “a virus particle” is intended to mean a type of virus particle and that the singular form of the term may encompass a large number of individual virus particles. Herein, the term “virus particle” may be used interchangeably with the terms “vector” and “capsid”, respectively, as further defined below.

The term “vector” is herein used to denote a virus particle, normally a recombinant virus particle, which is intended for use to achieve gene transfer to modify specific cell type or tissue. A virus particle can for example be engineered to provide a vector expressing therapeutic genes. Several virus types are currently being investigated for use to deliver genetic material (e.g., genes) to cells to provide either transient or permanent transgene expression. These include adenoviruses, retroviruses (γ-retroviruses and lentiviruses), poxviruses, adeno-associated viruses (AAV), baculoviruses, and herpes simplex viruses. Herein, the term “vector” may be used interchangeably with the terms “virus particle” and “capsid”, respectively.

The term “capsid” means the shell of a virus particle. The capsid surrounds the core of the virus particle, and normally should comprise a viral genome. A modified (recombinant) capsid, as produced in an upstream process of manufacturing, is supposed to comprise a complete viral genome, which genome includes genetic material of interest for one or more applications, for example of interest for various therapeutic applications. However, owing to low packaging efficiency, assembled capsids do not always contain any genetic material or only encapsidate truncated genetic fragments, resulting in so-called empty capsids and partially filled capsids, respectively. These capsids possess no therapeutic function, yet they compete for binding receptors during the cell-mediated processes. This may diminish the overall therapeutic efficacy and trigger undesirable immune responses. As a result, tracking these capsids throughout the production process is crucial to ensure consistent product quality and a proper dosing response (Xiaotong Fu et al, Analytical Strategies for Quantification of Adeno-Associated Virus Empty Capsids to Support Process Development, Human gene therapy methods, 2019, 30(4): 144-152). In up to 20-30% of a population of virus particles artificially produced in a cell culture, the capsid is only partially filled with genetic material. Further, in up to as much as 98% of artificially produced virus particles, the capsid does not comprise any part of the viral genome at all, i.e., it is empty. However, generally between 80% to 90% of artificially produced virus particles have empty capsids, and best cases currently achieve as little as 50% empty capsids.

Herein, the term “capsid” may be used interchangeably with the terms “vector” and “virus particle”, respectively. In the context of the present disclosure, a capsid may or may not comprise genetic material.

The term “genetic material of interest” is intended to mean genetic material which in the field of bioprocessing is considered relevant and valuable to get produced by viral replication and to purify such that it can be used in various applications, such as, but not limited to, therapeutic applications.

As a non-limiting example, genetic material of interest may comprise a therapeutically relevant genetic material, such as a therapeutically relevant nucleotide sequence.

The term “capsid fully packaged with genetic material” is herein used to denote a capsid which has been correctly produced (by the host cell), or in other words,
a capsid which comprises a complete viral genome, or in other words,
a capsid comprising 100% of its viral genome, or in other words,
a capsid comprising a functional viral genome.

The viral genome includes a genetic insert, comprising genetic material of interest, as defined elsewhere herein.

A capsid which comprises a complete viral genome may herein alternatively be called a “full capsid” or a “fully packaged capsid”. The terms “full capsid”, “fully packaged capsid”, and “capsid fully packaged with genetic material” may be used interchangeably throughout this text.

The term “capsid not fully packaged with genetic material” is herein used to denote a capsid which has not been correctly produced (by the host cell), or in other words,
a capsid which does not comprise a complete viral genome, or in other words,
a capsid which comprises less than 100% of its viral genome.

A capsid which is not fully packaged with genetic material is either partially filled with genetic material or is not filled with any genetic material at all.

The term “capsid not fully packaged with genetic material” encompasses the terms “partially filled capsid” and “empty capsid”, as defined below.

A “partially filled capsid” is herein defined as a capsid which comprises parts of its viral genome, such as defective parts of its viral genome, or in other words,
a capsid which comprises a partial viral genome, or in other words,
a capsid which comprises a non-complete viral genome, or in other words,
a capsid which comprises a defective viral genome, or in other words,
a capsid which comprises more than 0% and less than 100% of the complete viral genome, such as from about 1% to about 99%, such as from about 5% to about 95%, such as from about 10% to about 90%, or such as about 1%, about 5%, about 10%, about 20%, about 30%, about 40%, about 50%, about 60%, about 70%, about 80%, about 90%, about 95%, or about 99%, of the complete viral genome. Since a partially filled capsid is an incorrectly produced capsid, it is desirable to separate and remove as many as possible of the partially filled capsids from a population of capsids, before putting the population of capsids to use in its intended application, e.g., a therapeutic application. Herein, a partially filled capsid may alternatively be called an “intermediate capsid”.

An “empty capsid” is herein defined as a capsid which does not comprise any part of its viral genome, i.e., which comprises 0% of its viral genome, or in other words,
a capsid which is not filled with any genetic material at all. Thus, an empty capsid does not comprise any genetic material of interest. Consequently, it is desirable (and sometimes required, e.g., due to clinical regulations) to separate and remove as many as possible of the empty capsids from a population of capsids, before putting the population of capsids to use in its intended application, e.g., a therapeutic application.

Before putting a population of virus particles to use in its intended application, e.g., a therapeutic application, it is desirable (sometimes even required, e.g., due to clinical regulations) to enrich the full capsids, i.e., to increase the percentage of full capsids at the expense of the percentage of partially filled capsids and empty capsids.

The percentage of full capsids and empty capsids in a population of capsids can be estimated or analyzed with several methods known in the art. Some of these methods are briefly described below:

1: A260:280 in chromatogram will give an estimation of percentage full capsids present in peaks (ratio 1-1.5 indicate enriched in full capsids, ratio 0.5-0.7 is containing mainly empty capsids).

2. qPCR:ELISA ratio. qPCR quantifies viral genomes and ELISA quantifies total viral particles. A ratio of 2 assays with variation is less accurate and will be uncertain. Requires orthogonal analysis for confirmation (see below, 3,4 or 5).

3. Analytical anion exchange separating full and empty capsids (A260:280 ratio and peak area to calculate the percentage). Accuracy dependent of peak definition.

4. Analytical ultracentrifugation (AUC). Detects and quantifies particles of different density (corresponding to full, partially filled, and empty capsids). This is currently known as the “golden standard” in the art. However, ultracentrifugation is not scalable and thus is not suitable for analysis of large-scale batches of capsids.

5. Transmission electron microscopy (TEM). Image analysis counting particles (full, partially filled, and empty capsids). May introduce artifacts from sample preparation.

Some methods for estimating or analyzing the percentage of full capsids and empty capsids in a population of capsids are described in more detail in Xiaotong Fu et al, Analytical Strategies for Quantification of Adeno-Associated Virus Empty Capsids to Support Process Development, Human gene therapy methods, 2019, 30(4): 144-152, which is hereby incorporated by reference herein.

It is to be understood that the term “liquid sample” as used herein encompasses any type of sample obtainable from a cell culture, or from a fluid originating from a cell culture which fluid is at least partly purified, by any means of separation and purification.

The term “separation matrix” is used herein to denote a material comprising a support to which one or more ligands comprising functional groups have been coupled. The functional groups of the ligand(s) bind compounds herein also called analytes, which are to be separated from a liquid sample and/or which are to be separated from other compounds present in the liquid sample. A separation matrix may further comprise a compound which couples the ligand(s) to the support. The terms “linker”, “extender”, and “surface extender” may be used to describe such a compound, as further described below. The term “resin” is sometimes used for a separation matrix in this field. The terms “chromatography material” and “chromatography matrix” are used herein to denote a type of separation matrix.

The term “surface” herein means all external surfaces and includes in the case of a porous support outer surfaces as well as pore surfaces.

Herein, the term “strong anion exchange chromatography material” is intended to mean a chromatography material which comprises a ligand comprising a quaternized amine group. A quaternary amine group is a strong anion exchange group, which is always positively charged irrespective of to which pH it is subjected. For DEAE-based types of chromatography materials, the degree of quaternization of the amine group may vary among the amine groups included in a chromatography material. A degree of quaternization of the amine group of from about 12% to about 100% globally in a chromatography material is generally considered to result in a chromatography material which behaves like a strong, or at least partially strong, anion exchange chromatography material since these at least 12% of all amine groups are always charged. In contrast to quaternized amine groups, almost all other ionic exchange groups are weak, i.e., their charge varies from fully charged to not charged within a reasonable range of pH used (such as pH 2-11) and having a neutral charge (same amount of + and — charges) at pl.

Capto Q (Cytiva, Sweden) is a non-limiting example of a strong anion exchange chromatography material having about 100% quaternized amine groups. Capto DEAE (Cytiva, Sweden) is a non-limiting example of a strong, or partially strong, anion exchange chromatography material having a degree of quaternization of the amine groups of about 15%.

The separation matrix may be contained in any type of separation device, as further defined elsewhere herein. As a non-limiting example, a chromatography material may be packed in a chromatography column, before adding a liquid sample to the chromatography material being contained in the chromatography column.

In this context, “ligand” is a molecule that has a known or unknown affinity for a given analyte and includes any functional group, or capturing agent, immobilized on its surface, whereas “analyte” includes any specific binding partner to the ligand. The term “ligand” may herein be used interchangeably with the terms “specific binding molecule”, “specific binding partner”, “capturing molecule” and “capturing agent”. Herein, the molecules in a liquid sample which interact with a ligand are referred to as “analyte”. The analytes of interest according to the present disclosure are adeno-associated virus capsids, more particularly adeno-associated virus capsids either fully packaged or not fully packaged with genetic material. Consequently, herein the terms “analyte”, “adeno-associated virus capsid” and “capsid” may be used interchangeably.

In the herein disclosed method for separating fully packaged capsids from not fully packaged capsids, the chromatography material used comprises a linker connecting the ligand to the support, i.e., the coupling of the ligand to the support is provided by introducing a linker between the support and ligand. The coupling may be carried out following any conventional covalent coupling methodology such as by use of epichlorohydrin; epibromohydrin; allyl-glycidylether; bis-epoxides such as butanedioldiglycidylether; halogen-substituted aliphatic substances such as di-chloro-propanol; and divinyl sulfone. Other non-limiting examples of suitable linkers are: polyethylene glycol (PEG) having 2-6 carbon atoms, carbohydrates having 3-6 carbon atoms, and polyalcohols having 3-6 carbon atoms. These methods are all well known in the art and easily carried out by the skilled person.

The ligand is preferably coupled to the support via a longer linker molecule, also known as a “surface extender”, or simply “extender”. Extenders are well known in this field, and commonly used to sterically increase the distance between ligand and support. Extenders are sometimes denoted tentacles or flexible arms. For a more detailed description of possible chemical structures, see for example U.S. Pat. No. 6,428,707, which is hereby included herein by reference. In brief, the extender may be in the form of a polymer such as a homo- or a copolymer. Hydrophilic polymeric extenders may be of synthetic origin, i.e., with a synthetic skeleton, or of biological origin, i.e., a biopolymer with a naturally occurring skeleton. Typical synthetic polymers are polyvinyl alcohols, polyacryl- and polymethacrylamides, polyvinyl ethers etc. Typical biopolymers are polysaccharides, such as starch, cellulose, dextran, agarose. The results described in Example 1 herein surprisingly show that a chromatography material comprising a surface extender provides an improved separation of full AAV capsids from empty AAV capsids compared to the same chromatography material not including a surface extender.

The term “eluent” is used in its conventional meaning in this field, i.e., a buffer of suitable pH and/or ionic strength to release one or more compounds from a separation matrix.

The term “eluate” is used in its conventional meaning in this field, i.e., the part(s) of a liquid sample which are eluted from a chromatography column after having loaded the liquid sample onto the chromatography column.

As mentioned above, in the method for separating fully packaged capsids from not fully packaged capsids, the liquid sample which is added to a chromatography material in step (a) comprises adeno-associated virus capsids of a purity of at least 90%, such as 90%, 91%, 92%, 93%, 94%, 95%, 96%, 97%, 98%, or 99% and of a concentration of at least 10¹², such as 10¹³, 10¹⁴, or 10¹⁵, adeno-associated virus capsids/ml, of which at least 10%, such as 15%, 20%, 25%, 30%, 40%, 50%, 60%, 70%, or 80%, of the adeno-associated virus capsids are adeno-associated virus capsids fully packaged with genetic material. With regard to the purity of adeno-associated capsids in the liquid sample, a purity of at least 90%, such as up to 99%, is intended to mean that at least 90%, such as up to 99%, of the biological material in the liquid sample is represented by adeno-associated capsids (including full, empty, and partially filled capsids) while the remaining up to 10%, such as 1%, is represented by host cell protein and DNA.

The aim of step (b) of the above-disclosed method is to obtain fully packaged capsids of a purity which is as high as possible. A person skilled in the art readily understands that this may be achieved by applying various different separation conditions. Non-limiting examples of separation conditions to obtain fully packaged capsids of a purity as high as possible include separation conditions which allow binding of not fully packaged capsids to the chromatography material, while:
(i) allowing fully packaged capsids to substantially flow through the chromatography material (i.e., fully packaged capsids substantially not binding to the chromatography material), or
(ii) allowing fully packaged capsids to bind to the chromatography material followed by eluting them from the chromatography material. It is to be understood that in the bind-elute process described in item (ii), the fully packaged capsids may be eluted from the chromatography material before or after not fully packaged capsids, depending on which separation conditions are applied.

As mentioned above, there are small differences between fully packaged capsids and not fully packaged capsids in relation to several parameters relevant for purification, e.g., their isoelectric point. This often leads to (at least partial) co-elution of fully packaged and not fully packaged capsids. Accordingly, realistically, the adeno-associated virus capsids eluted in step (b) of the above-disclosed method will not be completely separated into full, empty, and partially filled capsids. However, there will be eluate fractions which comprise a substantially higher percentage of full capsids than in the liquid sample added to the chromatography material in step (a). More particularly, as disclosed above, the adeno-associated virus capsids eluted in step (b), i.e., adeno-associated virus capsids fully packaged with genetic material, are eluted into eluate fractions, which eluate fractions combined comprise at least 50%, such as 55%, 60%, 65%, 70%, 75%, 80%, 85%, or 90%, of the adeno-associated virus capsids of the liquid sample added in step (a), of which at least 60%, such as 65%, 70%, 75%, 80%, 85%, or 90%, of the adeno-associated virus capsids are fully packaged with genetic material. Non-limiting examples of recovery and purification of full capsids achieved by the presently disclosed method are a recovery of at least 50% of the capsids of the liquid sample added in step (a), of which at least 60% are full capsids, such as a recovery of at least 70% of the capsids of the liquid sample added in step (a), of which at least 80% are full capsids. In Example 1 described further below, the results show a recovery of at least 60% of capsids from harvest, of which at least 60% are full capsids. At present, there is no large-scale method publicly available which gives as high recovery and purification as the herein disclosed methods.

As mentioned above, the chromatography material applied in the presently disclosed method comprises a strong, or partially strong, anion exchange chromatography material comprising a support and a ligand for binding to the adeno-associated virus capsids.

The ligand of the strong anion exchange chromatography material may be defined by the following Formula I:

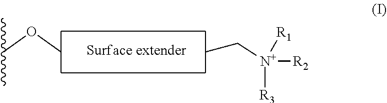

wherein
R₁ is selected from C1-C3 alkyl, and R₂ and R₃ are independently selected from C1-C3 alkyl, CH2OH, and CH2CHOHCH3.

As a non-limiting example, each of R₁, R₂, and R₃ is CH3.

There are currently available chromatography materials comprising a ligand defined by Formula I, wherein each of R₁, R₂, and R₃ is CH3; e.g., a chromatography material made available under the name Capto Q, provided by Cytiva, Sweden (www.cytivalifesciences.com). Capto Q further comprises dextran as surface extender and is a chromatography medium for high-resolution polishing steps in industrial purification processes, e.g., for purification of monoclonal antibodies. However, previously it has not been used for separation of fully packaged adeno-associated capsids from not fully packaged adeno-associated capsids.

According to another non-limiting example, R₁ and R₂ are ethyl, and R₃ is methyl.

According to yet another non-limiting example, R₁ and R₂ are methyl, and R₃ is CH2CHOHCH3.

The density of ligand defined by Formula I may be from about 60 to about 500 μmol, such as from about 160 to about 350 μmol, such as from about 160 to about 220 μmol, of ligand per ml of the strong anion exchange chromatography material.

Alternatively, the ligand of the strong, or partially strong, anion exchange chromatography material may be defined by the following Formula II:

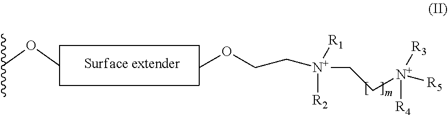

wherein:
m is an integer of from 1 to 3;
R₁ and R₂ are independently selected from a C1-C3 alkyl; R₃, and R₄ are independently selected from C1-C3 alkyl and CH2CHOHCH3; and R₅ is selected from hydrogen, a C1-C3 alkyl and CH2CHOHCH3; provided that if m is 1, the ligand of the strong, or partially strong, anion exchange chromatography material is defined by the following Formula III:

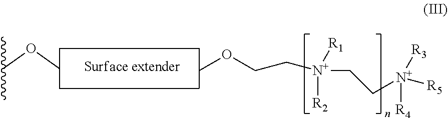

wherein n is an integer of from 0 to 3;
provided that if n is 0, R₃ and R₄ are independently selected from C1-C3 alkyl, and R₅ is hydrogen or CH2CHOHCH3.

As a non-limiting example, the ligand is defined by Formula III and comprises a combination of two or more of the following structures (i)-(iv):
(i) n is 0; R₃ and R₄ are ethyl; and R₅ is hydrogen or CH2CHOHCH3;
(ii) n is 1; R₁, R₂, R₃, R₄ are ethyl; and R₅ is hydrogen or CH2CHOHCH3;
(iii) n is 2; each R₁ and R₂ is ethyl; R₃ and R₄ is ethyl; and R₅ is hydrogen or CH2CHOHCH3;
(iv) n is 3; each R₁ and R₂ is ethyl; R₃ and R₄ is ethyl; and R₅ is hydrogen or CH2CHOHCH3.

One currently available chromatography material comprising a ligand defined by Formula III and comprising a combination of the above-mentioned structures (i)-(iv) is the chromatography resin called Capto DEAE (Cytiva, Sweden). Capto DEAE further comprises dextran as surface extender and is a chromatography medium for high-resolution polishing steps in industrial purification processes, e.g., for purification of monoclonal antibodies. However, previously it has not been used for separation of fully packaged adeno-associated capsids from not fully packaged adeno-associated capsids.

According to another non-limiting example, the ligand is defined by Formula III, wherein m is 1; n is 1, 2, or 3; each R₁, R₂, R₃, and R₄ is methyl; and R₅ is hydrogen.

According to yet another non-limiting example, the ligand is defined by Formula III, wherein m is 1; n is 1, 2, or 3; each R₁, R₂, R₃, and R₄ is methyl; and R₅ is CH2CHOHCH3.

According to another non-limiting example, the ligand is defined by Formula III, wherein m is 1 and the ligand comprises a combination of two or more of the following structures (i)-(iv):
(i) n is 0; R3 and R4 are methyl; R5 is hydrogen or CH2CHOHCH3;
(ii) n is 1; R1, R2, R3, and R4 are methyl; R5 is hydrogen or CH2CHOHCH3;
(iii) n is 2; each R1 and R2 is methyl; R3 and R4 is methyl; R5 is hydrogen or CH2CHOHCH3;
(iv) n is 3; each R1 and R2 is methyl; R3 and R4 is methyl; R5 is hydrogen or CH2CHOHCH3.

The density of ligand defined by Formula II or Formula III may be from about 60 to about 500 μmol, such as from about 160 to about 350 μmol, such as from about 290 to about 350 μmol, of ligand per ml of the strong, or partially strong, anion exchange chromatography material.

As described above, the chromatography material comprises a surface extender connecting the ligand to the support, wherein the surface extender is a polymer, wherein the polymer is selected from:
(i) a polymer having a naturally occurring skeleton, such as a polysaccharide, such as starch, cellulose, dextran, or agarose; and
(ii) a polymer having a synthetic skeleton, such as a polyvinyl alcohol, a polyacrylamide, a polymethacrylamide, or a polyvinyl ether.

As a non-limiting example, the surface extender is dextran. The dextran may have a molecular weight of from about 10 to about 2000 kDa, such as about 10, 40, 70, 250, 750, or 2000 kDa, such as 40 kDa. The density of dextran may be from about 5 to about 30 mg dextran per ml of the chromatography material. It is to be understood that the amount of dextran immobilized on the chromatography material may vary, for example depending on the molecular weight of the dextran immobilized. Normally, decreasing amounts are required for increasing molecular weights of dextran.

Steps (a) and (b) of the above-disclosed method may comprise applying a buffer having a pH of from about 6.0 to about 10.5, such as from about 7.0 to about 10.0, such as from about 7.5 to about 9.5, or about 6.0, 6.5, 7.0, 7.5, 8.0, 8.5, 9.0, 9.5, 10.0, or 10.5. According to non-limiting examples, as described in Example 1 below, a pH of about 9.5 may be applied for a chromatography material comprising a ligand defined by Formula I. Further, a pH of about 7.5 may be applied for a chromatography material comprising a ligand defined by Formula II or Formula III.

Said buffer is suitably selected from buffers generally recommended for anion exchange chromatography and may for example comprise tris(hydroxymethyl)amino-methane (i.e., Tris), 1,3-bis(tris(hydroxymethyl)methylamino) propane (i.e., bis-Tris propane), triethanolamine, N-methyldiethanolamine, Diethanolamine, 1,3-diaminopropane, or ethanolamine. A person skilled in the art is able to choose a suitable concentration for any one of the above-listed buffers.

In the above-disclosed method, step (b) may comprise applying a buffer, optionally one of the buffers mentioned above, wherein the buffer comprises a compound which improves separation between capsids fully packaged with genetic material and capsids not fully packaged with genetic material. This compound may or may not be present in a buffer applied in step (a). Without being bound by theory, such a compound may for example improve separation by influencing interactions between capsid and ligand or interactions between capsid and capsid. Said compound which improves separation may for example be selected from a carbohydrate, a divalent metal ion, and a detergent.

Where said compound which improves separation is a carbohydrate, it may for example be selected from sucrose, sorbitol, and a polysaccharide.

Where said compound which improves separation is a divalent metal ion, it may for example be selected from Mg²⁺, Fe²⁺, and Mn²⁺. The metal ion may be present in the form of a salt, optionally in combination with for example chloride ions or sulphate ions. A non-limiting example of a suitable metal salt to include in the buffer of step (b) is MgCl₂. Non-limiting examples of suitable concentrations of MgCl₂ include from about 0.5 to about 30 mM of MgCl₂, such as from about 1 to about 20 mM, such as from about 2 to about 10 mM, or about 0.5, 1.0, 1.5, 2.0, 2.5, 3, 4, 5, 6, 7, 8, 9, 10, 11, 12, 13, 14, 15, 16, 17, 18, 19, 20, 25, or 30 mM, of MgCl₂.

Where said compound which improves separation is a detergent, it may for example be selected from poloxamer, such as poloxamer 188 or Pluronic™ F68, and polysorbate, such as Tween 20 or Tween 80.

In the above-described method, step (b) may comprise applying a buffer, optionally one of the buffers mentioned above, wherein the buffer comprises a compound which may help eluting capsids bound to the chromatography material. This compound is not present in a buffer applied in step (a). Non-limiting examples of such a compound is a salt, such as a salt of a monovalent metal ion. More particularly, the salt may be a kosmotropic salt. Salts in water solvent are defined as kosmotropic (order-making) if they contribute to the stability and structure of water-water interactions. In contrast, chaotropic (disorder-making) salts have the opposite effect, disrupting water structure, increasing the solubility of nonpolar solvent particles, and destabilizing solute aggregates. Kosmotropes cause water molecules to favorably interact, which in effect stabilizes intramolecular interactions in macromolecules such as proteins (Moelbert S et al). A scale can be established for example by referring to the Hofmeister series, or lyotropic series, which is a classification of ions in order of their ability to salt out or salt in proteins (Hyde A et al).

More particularly, the kosmotropic salt may comprise (i) an anion selected from a group consisting of CO₃²⁻, SO₄²⁻, S₂O₃²⁻, H₂PO₄⁻, HPO₄²⁻, acetate⁻, citrate⁻, and Cl⁻, and (ii) a cation selected from a group consisting of NH₄*, K*, Na*, and Li*. In a currently preferred embodiment, the salt is sodium acetate (NaOAc). Non-limiting examples of suitable concentrations of NaOAc include from about 5 mM to about 500 mM, such as about 5, 10, 20, 30, 40, 50, 60, 70, 80, 90, 100, 150, 200, 250, 300, 350, 400, 450, or 500 mM. However, it is to be understood that other salts consisting of a combination an anion as listed under (i) and a cation as listed under (ii) may alternatively be used to elute the capsids. Non-limiting examples are NaCl, LiCl, KCl, or other equivalent metal salt suitable to use for salt elution, as is well known in the art. Non-limiting examples of suitable concentrations of NaCl include from about 5 mM to about 2M of NaCl, such as about 5, 10, 20, 30, 40, 50, 100, 150, 200, 250, 300, 350, 400, 450, 500, 600, 700, 800, 900, 1000, 1500, or 2000 mM, of NaCl. Further, step (b) may comprise applying a gradient of such a compound to improve elution of the adeno-associated virus capsids fully packaged with genetic material from the chromatography material. Such a gradient may be a linear gradient or a step gradient, or a combination thereof.

A non-limiting example of a suitable buffer to be applied in step (b) may comprise 20 mM bis-Tris propane (BTP), pH 9.5 (for ligand defined by Formula I) or pH 7.5 (for ligand defined by Formula II or III), 2 mM MgCl₂, 1% sucrose, and 0.1% Poloxamer 188.

The chromatography material applied in the herein disclosed methods comprises a support to which the ligand is coupled. The support may be made from an organic or inorganic material and may be porous or non-porous. In one embodiment, the support is prepared from a native polymer, such as cross-linked carbohydrate material, e.g. agarose, agar, cellulose, dextran, chitosan, konjac, carrageenan, gellan, alginate, pectin, starch, etc. The native polymer supports are easily prepared and optionally cross-linked according to standard methods, such as inverse suspension gelation (S Hjerten: Biochim Biophys Acta 79(2), 393-398 (1964). In an especially advantageous embodiment, the support is a kind of relatively rigid but porous agarose, which is prepared by a method that enhances its flow properties, see e.g. U.S. Pat. No. 6,602,990 (Berg). In an alternative embodiment, the support is prepared from a synthetic polymer or copolymer, such as cross-linked synthetic polymers, e.g. styrene or styrene derivatives, divinylbenzene, acrylamides, acrylate esters, methacrylate esters, vinyl esters, vinyl amides etc. Such synthetic polymers are easily prepared and optionally cross-linked according to standard methods, see e.g. “Styrene based polymer supports developed by suspension polymerization” (R Arshady: Chimica e L'Industria 70(9), 70-75 (1988)). Native or synthetic polymer supports are also available from commercial sources, such as Cytiva, Sweden, for example in the form of porous particles. In yet an alternative embodiment, the support is prepared from an inorganic polymer, such as silica. Inorganic porous and non-porous supports are well known in this field and easily prepared according to standard methods.

The support of the chromatography material may be in the form of particles, such as substantially spherical, elongated or irregularly formed particles.

Where the chromatography material is in the form of particles, the particles may be particles having a homogeneous porosity and being at least partly permeable to adeno-associated virus capsids.

Herein, the term “homogeneous porosity” is intended to mean that a particle having a homogeneous porosity has a homogeneous porosity throughout its entire structure or volume, such that each particle is at least partly permeable to adeno-associated virus capsids throughout its entire structure or volume. In other words, a particle having a homogeneous porosity has a porosity which permits adeno-associated virus capsids to diffuse, completely or at least partly, through its pores, throughout the entire structure or volume of the particle.

Adeno-associated viruses are approx. 20-25 nm in diameter. Since a capsid is the shell of a virus particle, and since adeno-associated viruses do not have a lipoprotein bilayer envelope surrounding the capsid, the size of an adeno-associated virus capsid is approx. 20-25 nm in diameter.

Accordingly, where the chromatography material is in the form of particles having a homogeneous porosity and being at least partly permeable to adeno-associated virus capsids, each particle may suitably comprise pores of a diameter which is >25 nm, i.e., larger than the diameter of the adeno-associated virus capsids to be separated, thereby enabling diffusion of capsids within the entire particle. It is to be understood that for the specific purposes of the present disclosure, i.e., to separate adeno-associated virus capsids, a diameter >25 nm may be of any size >25 nm, including but not limited to 30, 50, 75, 100, 150, or 200 nm.

Further, it is to be understood that a particle having a homogeneous porosity throughout its entire structure or volume nevertheless may comprise pores of different sizes, both pores that are large enough to easily allow capsids to diffuse within the particle and pores that are small enough not to allow diffusion of capsids. This diversity of pore size can be measured by the diffusion coefficient of a molecule of a well-defined molecular weight and hydrodynamic size. As a non-limiting example, dextran, which has a molecular weight of 140-225 kDa or a hydrodynamic diameter of 20-25 nm (i.e., a diameter of the same size as adeno-associated virus capsids), can be used to evaluate the degree of diffusion of adeno-associated virus capsids within the pores of the particles.

The chromatography materials Capto Q and Capto DEAE, advantageously used in Example 1 herein, comprise a support in the form of substantially spherical particles or beads, which have a diameter of approx. 90 μm. This type of particle is a non-limiting example of a particle having a homogeneous porosity (i.e., throughout its entire structure or volume) and being at least partly permeable to adeno-associated virus capsids (i.e., throughout its entire structure or volume).

Suitable particle sizes of a chromatography material for use in the presently disclosed methods may be in a diameter range of 5-500 μm, such as 10-100 μm, e.g., 30-90 μm. In the case of essentially spherical particles, the average particle size may be in the range of 5-1000 μm, such as 10-500. In a specific embodiment, the average particle size is in the range of 10-200 μm. The skilled person in this field can easily choose the suitable particle size and porosity depending on the process to be used. For example, for a large-scale process, for economic reasons, a more porous but rigid support may be preferred to allow processing of large volumes, especially for the capture step. In chromatography, process parameters such as the size and the shape of the column will affect the choice. In an expanded bed process, the matrix commonly contains high density fillers, preferably stainless-steel fillers. For other processes other criteria may affect the nature of the matrix.

The chromatography material may be dried, such as dried particles which upon use are soaked in liquid to retain their original form. For example, such a dried chromatography material may comprise dried agarose particles.

The support of the chromatography material may alternatively take any other shape conventionally used in separation, such as monoliths, filters or membranes, capillaries, chips, nanofibers, surfaces, etc.

Where the support of the chromatography material comprises a monolith, a suitable pore diameter in the monolith for the purpose of separating adeno-associated virus capsids ranges from a minimum pore diameter of >25 nm, i.e., larger than the diameter of the capsids to be separated, and up to a maximum pore diameter of about 5 μm, such as about 0.5, 1.0, 2.0, 3.0, 4.0, or 5.0 μm.

Where the support of the chromatography material comprises nanofibers, such nanofibers may for example comprise electrospun polymer nanofibers. When in use, such nanofibers form a stationary phase comprising a plurality of pores through which a mobile phase can permeate.

The support of the chromatography material may comprise a membranous structure, such as a single membrane, a pile of membranes or a filter. The membrane may be an adsorptive membrane. Where the support of the chromatography material comprises a membranous structure, a suitable pore diameter in the membranous structure for the purpose of separating adeno-associated virus capsids ranges from a minimum pore diameter of >25 nm, i.e., larger than the diameter of the capsids to be separated, and up to a maximum pore diameter of about 5 μm, such as about 0.5, 1.0, 2.0, 3.0, 4.0, or 5.0 μm. Where the chromatography material comprises a membranous structure, such membranous structure may for example comprise a nonwoven web of polymer nanofibers.

Non-limiting examples of suitable polymers may be selected from polysulfones, polyamides, nylon, polyacrylic acid, polymethacrylic acid, polyacrylonitrile, polystyrene, and polyethylene oxide, and mixtures thereof.

Alternatively, the polymer may be a cellulosic polymer, such as selected from a group consisting of cellulose and a partial derivative of cellulose, particularly cellulose ester, cross-linked cellulose, grafted cellulose, or ligand-coupled cellulose. Cellulose fiber chromatography (known as Fibro chromatography; Cytiva, Sweden) is an ultrafast chromatography purification for short process times and high productivity, which utilizes the high flow rates and high capacities of cellulose fiber. Where the support of the chromatography material comprises cellulose fibers such as Fibro, a suitable pore diameter in the cellulose fiber for the purpose of separating adeno-associated virus capsids ranges from a minimum pore diameter of >25 nm, i.e., larger than the diameter of the capsids to be separated, and up to a maximum pore diameter of about 5 μm, such as about 0.1, 0.2, 0.5, 1.0, 2.0, 3.0, 4.0, or 5.0 μm.

The term “membrane chromatography” has its conventional meaning in the field of bioprocessing. In membrane chromatography there is binding of components of a fluid, for example individual molecules, associates or particles, to the surface of a solid phase in contact with the fluid. The active surface of the solid phase is accessible for molecules by convective transport. The advantage of membrane adsorbers over packed chromatography columns is their suitability for being run with much higher flow rates. This is also called convection-based chromatography. A convection-based chromatography matrix includes any matrix in which application of a hydraulic pressure difference between the inflow and outflow of the matrix forces perfusion of the matrix, achieving substantially convective transport of substance(s) into the matrix or out of the matrix, which is effected very rapidly at a high flow rate. Convection-based chromatography and membrane adsorbers are described in for example US20140296464A1, US20160288089A1, WO2018011600A1, WO2018037244A1, WO2013068741A1, WO2015052465A1, U.S. Pat. No. 7,867,784B2, hereby incorporated by reference in their entirety.

In the herein disclosed method for separating adeno-associated virus capsids fully packaged with genetic material from adeno-associated virus capsids not fully packaged with genetic material, the chromatography material used, the chromatography material referred to in steps (a) and (b) of the method may advantageously be a polishing chromatography material, meaning that the chromatography material is applied in a polishing step.

The term “polishing step” refers in the context of liquid chromatography to a final purification step, wherein trace impurities are removed to leave an active, safe product. Impurities removed during the polishing step are often conformers of the target molecule, i.e., forms of the target molecule having particular molecular conformations, or suspected leakage products. A polishing step may alternatively be called “secondary purification step”.

Further, the liquid sample added in step (a) of the herein disclosed method for separating adeno-associated virus capsids fully packaged with genetic material from adeno-associated virus capsids not fully packaged with genetic material may advantageously be a pre-purified liquid sample.

The present disclosure further provides a method for separating fully packaged adeno-associated virus capsids from not fully packaged adeno-associated virus capsids, comprising performing the method as shown in FIG. 1 and as described in detail above, the method further comprising a step (a1) which comprises pre-purifying adeno-associated virus capsids by separating adeno-associated virus capsids from an adeno-associated virus capsid-containing cell culture harvest, as illustrated in FIG. 2, thereby obtaining a pre-purified liquid sample comprising adeno-associated virus capsids, before adding said pre-purified liquid sample comprising adeno-associated virus capsids to the chromatography material according to step (a) of the method as shown in FIG. 1.

Such a pre-purifying step (a1) may alternatively be called a “capture step” and refers in the context of liquid chromatography to the initial step(s) of a separation procedure. Most commonly, a capture step includes clarification (e.g. by filtration, centrifugation, or precipitation), and normally also concentration and/or stabilisation of the sample, and a significant purification from soluble impurities, for example by applying chromatography after the clarification, concentration, and stabilisation of sample. After the capture step, an intermediate purification may follow, which further reduces remaining amounts of impurities such as host cell proteins, DNA, viruses, endotoxins, nutrients, components of a cell culture medium, such as antifoam agents and antibiotics, and product-related impurities, such as aggregates, misfolded species, and aggregates.

Such a pre-purifying step may comprise subjecting the adeno-associated virus capsid-containing cell culture harvest to one or more of the following non-limiting examples of purification methods:
(i) affinity chromatography,
(ii) ion exchange chromatography,
(iii) precipitation or tangential flow filtration (TFF), followed by size-exclusion chromatography, such as by use of for example Capto Core 400 chromatography material (Cytiva, Sweden), which combines flow-through of the capsids with binding of impurities to the chromatography material,
(iv) TFF followed by ion exchange chromatography, and
(v) TFF followed by ion exchange chromatography and Capto Core.

Non-limiting examples of chromatography materials suitable to apply in a pre-purifying step of the method illustrated in FIG. 2 and described above include affinity chromatography material, ion exchange chromatography material, and size-exclusion chromatography material, respectively. The chromatography material may be functionalized with a positively charged group, such as a quaternary amino, quaternary ammonium, or amine group, or a negatively charged group, such as a sulfonate or carboxylate group. The chromatography material may be functionalized with an ion exchanger group, an affinity peptide/protein-based ligand, a hydrophobic interaction ligand, an IMAC ligand, or a DNA based ligand such as Oligo dT.

Herein, the term “cell culture” refers to a culture of cells or a group of cells being cultivated, wherein the cells may be any type of cells, such as bacterial cells, viral cells, fungal cells, insect cells, or mammalian cells. A cell culture may be unclarified, i.e., comprising cells, or may be cell-depleted, i.e., a culture comprising no or few cells but comprising biomolecules released from the cells before removing the cells. Further, an unclarified cell culture may comprise intact cells, disrupted cells, a cell homogenate, and/or a cell lysate.

The term “cell culture harvest” is used herein to denote a cell culture which has been harvested and removed from the vessel or equipment, in which the cells have been cultivated.

The term “separation device” has its conventional meaning in the field of bioprocessing and is to be understood as encompassing any type of separation device which is capable of and suitable for separating and purifying compounds from a fluid containing by-products from the production of the compounds. A separation device may comprise a separation matrix, as further defined elsewhere herein.

Non-limiting examples of separation devices suitable for use in the polishing step according to the presently disclosed method include chromatography columns and membrane devices, as further described elsewhere herein. Such separation devices may suitably comprise chromatography material in the form of a strong, or partially strong, anion exchange chromatography material comprising a ligand as defined by Formula I, II or III, as described in detail elsewhere herein.

Non-limiting examples of separation devices suitable for use in a capture step, or pre-purification step, as described herein, are filtration apparatuses, chromatography columns and membrane devices. Chromatography columns suitable for use in the capture step may for example be packed with affinity chromatography material, ion exchange chromatography material, mixed mode chromatography material or hydrophobic interaction chromatography material.

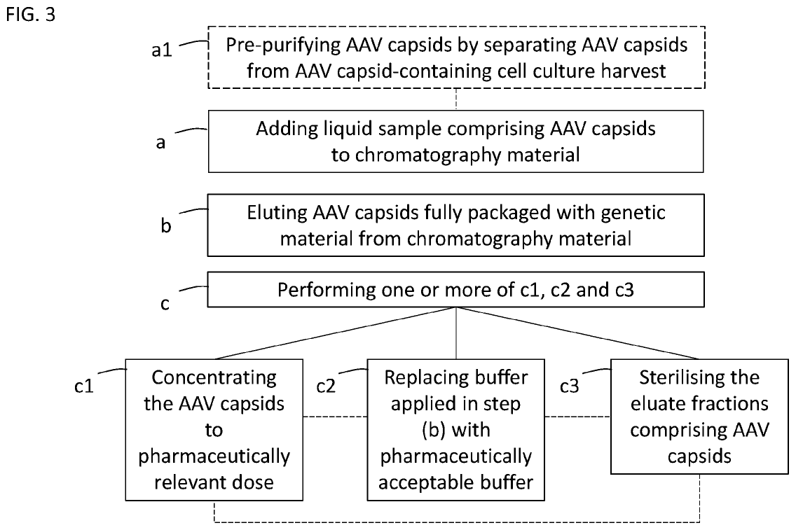

As illustrated in FIG. 3, the herein disclosed method for separating fully packaged adeno-associated virus capsids from not fully packaged adeno-associated virus capsids may further comprise subjecting the eluate fractions comprising adeno-associated virus capsids fully packaged with genetic material, eluted in step (b) of the method as described above, to one or more of the following steps:
c1) concentrating the adeno-associated virus capsids to a pharmaceutically relevant dose,
c2) replacing a buffer applied in step (b) of the method with a pharmaceutically acceptable buffer, and/or
c3) sterilizing the eluate fractions comprising adeno-associated virus capsids, thereby obtaining a pharmaceutical composition comprising adeno-associated virus capsids.

A person skilled in the art understands that the pharmaceutically relevant dose will depend on various factors such as, but not limited to, the disease or disorder to be treated as well as the weight and condition of the subject to be treated with a pharmaceutical composition.

Pharmaceutically acceptable buffers are well known in the art and can easily be chosen by the skilled person.

For the resulting composition to fulfil all regulatory requirements for pharmaceutical compositions, normally all of the above-listed three steps c1-c3 have to be performed.

In the above-disclosed method, the adeno-associated virus capsids may advantageously be capsids of adeno-associated virus serotype 8 (AAV8), adeno-associated virus serotype 5 (AAV5), adeno-associated virus serotype 1 (AAV1), adeno-associated virus serotype 2 (AAV2), adeno-associated virus serotype 4 (AAV4), adeno-associated virus serotype 6 (AAV6), adeno-associated virus serotype 7 (AAV7), adeno-associated virus serotype 9 (AAV9), or adeno-associated virus serotype 10 (AAV10), or a variant thereof.

The term “variant” in relation to an adeno-associated virus (AAV) serotype 1, 2, 4, 5, 6, 7, 8, or 10, as listed above, is intended to mean a modified or engineered AAV, in which the capsid structure has been modified to improve clinical performance, for example towards a specific target organ. As a non-limiting example, an AAV8 variant comprises capsid parts of AAV8 and may additionally comprise capsid parts of other AAV serotypes than AAV8, such as AAV5. However, an AAV8 variant as referred to herein must retain a significant structural similarity to a non-modified AAV8 capsid, such as retaining at least 50%, such as 60%, 70%, 80%, or 90%, of the external surface structure of a non-modified AAV8 capsid. This applies equally to a variant of AAV serotype 1, 2, 4, 5, 6, 7, or 10, as compared to a non-modified AAV serotype 1, 2, 4, 5, 6, 7, or 10, respectively. Further, as a non-limiting example, in the context of purification or separation of a variant of AAV8, a “variant” is herein defined as an adeno-associated virus which has a functionally equivalent binding capacity to the ligand of a specified chromatography material, compared to the binding capacity of the original AAV8 to said specified chromatography material. This applies equally to a variant of AAV serotype 1, 2, 4, 5, 6, 7, or 10, as compared to the original AAV serotype 1, 2, 4, 5, 6, 7, or 10, respectively. The specified chromatography material may, for example, be a strong, or partially strong, anion exchange chromatography material as disclosed in more detail elsewhere herein. A variant of an adeno-associated virus may for example be obtained by spontaneous mutation, or by engineered modification (i.e., obtained by human interaction), of one or more nucleotides of the genome of the adeno-associated virus.

According to a currently preferred embodiment, in the separation method as illustrated in FIG. 1, FIG. 2, and FIG. 3, respectively, the chromatography material is defined by Formula IV:

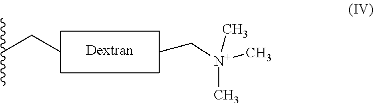

and the elution buffer of step (b) comprises sodium acetate. Said method may be applied for separation of AAV capsids of any serotype or variant as described above. In particular, the capsids to be separated may be capsids of the AAV9 serotype or a variant thereof.

The present disclosure also provides use of an anion exchange chromatography material comprising a support, a ligand, and a surface extender connecting the ligand to the support, and being defined by Formula IV:

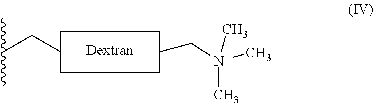

for separating adeno-associated virus capsids fully packaged with genetic material from adeno-associated virus capsids not fully packaged with genetic material,
comprising performing the following steps:
a. adding a liquid sample comprising adeno-associated virus capsids of a purity of at least 90% and of a concentration of at least 10¹² adeno-associated virus capsids/ml, of which at least 10% of the adeno-associated virus capsids are adeno-associated virus capsids fully packaged with genetic material, to the chromatography material;
b. eluting the adeno-associated virus capsids fully packaged with genetic material from the chromatography material;
wherein the adeno-associated virus capsids eluted in step (b) are eluted into eluate fractions, which eluate fractions combined comprise at least 50% of the adeno-associated virus capsids of the liquid sample added in step (a), of which at least 60% of the adeno-associated virus capsids are fully packaged with genetic material.

According to a currently preferred embodiment of said use, the elution buffer of step (b) comprises sodium acetate.

The adeno-associated virus capsids to be separated according to said use may be capsids of adeno-associated virus serotype 1 (AAV1), adeno-associated virus serotype 2 (AAV2), adeno-associated virus serotype 4 (AAV4), adeno-associated virus serotype 5 (AAV5), adeno-associated virus serotype 6 (AAV6), adeno-associated virus serotype 7 (AAV7), adeno-associated virus serotype 8 (AAV8), adeno-associated virus serotype 9 (AAV9), or adeno-associated virus serotype 10 (AAV10), or a variant thereof. In a particularly preferred embodiment of said use, the adeno-associated virus capsids are capsids of adeno-associated virus serotype 9 (AAV9) or a variant thereof.

The present disclosure further provides a method for preventing or treating a disease or disorder related to an organ or tissue in a subject, comprising administering to the subject a pharmaceutical composition comprising adeno-associated virus capsids obtained by performing the above-disclosed separation method comprising one or more of steps c1-c3 (as described in detail above), in which pharmaceutical composition the ratio of adeno-associated virus capsids fully packaged with genetic material to adeno-associated virus capsids not fully packaged with genetic material is at least 3:2, i.e., the number of full capsids is at least 1.5 times higher than the total number of empty capsids and partially filled capsids. Preferably, the ratio of adeno-associated virus capsids fully packaged with genetic material to adeno-associated virus capsids not fully packaged with genetic material is at least 2:1, such as 3:1, 4:1, or 5:1. That is, preferably the number of full capsids is at least 2, such as 3, 4, or 5, times higher than the total number of empty capsids and partially filled capsids.

Said method for preventing or treating a disease or disorder related to an organ or tissue may suitably comprise gene therapy. It is to be understood that the therapeutically relevant gene(s) or genetic material is present in the adeno-associated virus capsids fully packaged with genetic material comprised by the pharmaceutical composition which is to be administered to the subject.

In the above-disclosed method for preventing or treating a disease or disorder related to an organ or tissue, the adeno-associated virus capsids may advantageously be capsids of adeno-associated virus serotype 8 (AAV8), adeno-associated virus serotype 5 (AAV5), adeno-associated virus serotype 1 (AAV1), adeno-associated virus serotype 2 (AAV2), adeno-associated virus serotype 4 (AAV4), adeno-associated virus serotype 6 (AAV6), adeno-associated virus serotype 7 (AAV7), adeno-associated virus serotype 9 (AAV9), or adeno-associated virus serotype 10 (AAV10), or a variant thereof.

In the above-disclosed method for preventing or treating a disease or disorder related to an organ or tissue, the organ or tissue may optionally be selected from the central nervous system, heart, kidney, liver, lung, pancreas, photoreceptor cells, retinal pigment epithelium, skeletal muscle, and brain.

More particularly, the following combinations of AAV serotype and type of organ/tissue are contemplated:
(i) the capsids are selected from AAV1, AAV2, AAV4, AAV5, AAV8, and AAV10 capsids, and the organ or tissue is the central nervous system;
(ii) the capsids are selected from AAV1, and AAV8 capsids, and the organ or tissue is the heart;
(iii) the capsids are AAV2 capsids, and the organ or tissue is kidney;
(iv) the capsids are selected from AAV7, and AAV8 capsids, and the organ or tissue is liver;
(v) the capsids are selected from AAV4, AAV5, and AAV6 capsids, and the organ or tissue is lung;
(vi) the capsids are AAV8, and the organ or tissue is pancreas;
(vii) the capsids are selected from AAV2, AAV5, and AAV8 capsids, and the organ or tissue is photoreceptor cells;
(viii) the capsids are selected from AAV1, AAV2, AAV4, AAV5, and AAV8 capsids, and the organ or tissue is retinal pigment epithelium;
(ix) the capsids are selected from AAV1, AAV6, AAV7, and AAV8 capsids, and the organ or tissue is skeletal muscle;
(x) the capsids are AAV10 capsids and the organ or tissue is brain; or
(xi) the capsids are AAV9 capsids or a variant thereof, and the organ or tissue is selected from the central nervous system, heart, liver, lung, and skeletal muscle.

It is to be understood that the above-mentioned tissue types are non-limiting examples of organs and tissue types for which treatment by administration of a pharmaceutical composition comprising adeno-associated virus capsids, in particular capsids of adeno-associated virus serotype 8 (AAV8), adeno-associated virus serotype 5 (AAV5), adeno-associated virus serotype 1 (AAV1), adeno-associated virus serotype 2 (AAV2), adeno-associated virus serotype 4 (AAV4), adeno-associated virus serotype 6 (AAV6), adeno-associated virus serotype 7 (AAV7), adeno-associated virus serotype 9 (AAV9), or adeno-associated virus serotype 10 (AAV10), or a variant thereof, may be applicable.

A non-limiting example of a disease or disorder related to an organ or tissue is spinal muscular atrophy, for which a method of treatment or prevention may suitably be performed by administration of adeno-associated virus, in particular AAV5, AAV8, or AAV10, or a variant thereof.

Another non-limiting example of a disease or disorder related to an organ or tissue is inherited retinal dystrophy, for which treatment or prevention may suitably be performed by administration of adeno-associated virus, in particular AAV2, or a variant thereof.

Other non-limiting examples of a disease or disorder related to an organ or tissue are pancreatic tumors and metabolic disorders in liver, such as ornithine transcabamylase (OTC) deficiency, for which a method of treatment or prevention may suitably be performed by administration of adeno-associated virus, in particular AAV8, or a variant thereof.

A person of skill in the art understands that the pharmaceutical composition must be administered in a pharmaceutically effective amount or dose to the subject to achieve the desired medical effects. Amounts and doses which are pharmaceutically effective depend on various factors such as, but not limited to, the disease or disorder to be treated as well as the weight and condition of the subject to be treated.

The present disclosure further provides a composition comprising adeno-associated virus capsids obtained by performing the method for separating fully packaged adeno-associated virus capsids from not fully packaged adeno-associated virus capsids as described in detail above, in which composition the ratio of adeno-associated virus capsids fully packaged with genetic material to adeno-associated virus capsids not fully packaged with genetic material is at least 3:2, i.e., the number of full capsids is at least 1.5 times higher than the total number of empty capsids and partially filled capsids. Preferably, the ratio of adeno-associated virus capsids fully packaged with genetic material to adeno-associated virus capsids not fully packaged with genetic material is at least 2:1, such as 3:1, 4:1, or 5:1. That is, preferably the number of full capsids is at least 2, such as 3, 4, or 5, times higher than the total number of empty capsids and partially filled capsids.

The present disclosure also provides a pharmaceutical composition comprising adeno-associated virus capsids obtained by performing the above-disclosed separation method comprising one or more of steps c1-c3 (as described in detail above), in which pharmaceutical composition the ratio of adeno-associated virus capsids fully packaged with genetic material to adeno-associated virus capsids not fully packaged with genetic material is at least 3:2, i.e., the number of full capsids is at least 1.5 times higher than the total number of empty capsids and partially filled capsids. Preferably, the ratio of adeno-associated virus capsids fully packaged with genetic material to adeno-associated virus capsids not fully packaged with genetic material is at least 2:1, such as 3:1, 4:1, or 5:1. That is, preferably the number of full capsids is at least 2, such as 3, 4, or 5, times higher than the total number of empty capsids and partially filled capsids.

The above-described pharmaceutical composition may be for use in therapy, optionally for use in gene therapy.

In the above-described pharmaceutical composition for use in therapy, such as gene therapy, the adeno-associated virus capsids may advantageously be capsids of adeno-associated virus serotype 8 (AAV8), adeno-associated virus serotype 5 (AAV5), adeno-associated virus serotype 1 (AAV1), adeno-associated virus serotype 2 (AAV2), adeno-associated virus serotype 4 (AAV4), adeno-associated virus serotype 6 (AAV6), adeno-associated virus serotype 7 (AAV7), adeno-associated virus serotype 9 (AAV9), or adeno-associated virus serotype 10 (AAV10), or a variant thereof.

Further, the pharmaceutical composition may advantageously be for use in the prevention or treatment of a disease or a disorder related to an organ or tissue selected from the central nervous system, heart, kidney, liver, lung, pancreas, photoreceptor cells, retinal pigment epithelium, skeletal muscle, and brain.

Devices or compositions “comprising” one or more recited components may also include other components not specifically recited. The term “comprising” includes as a subset “consisting essentially of” which means that the device or composition has the components listed without other features or components being present. Likewise, methods “comprising” one or more recited steps may also include other steps not specifically recited.

The singular “a” and “an” shall be construed as including also the plural.

### EXAMPLES

### Example 1: Separation of AAV8 Capsids on Anion Exchange Chromatography Materials Using Magnesium Chloride and Increasing Sodium Chloride Linear Gradient

### Chromatography Materials

The following currently available anion exchange chromatography materials gave improved results, as described further below:

### Capto Q (Cytiva, Sweden):

Ligand: Quaternary amine

Particle size, d₅₀ᵥ: ˜90 μm

Matrix: Highly cross-linked agarose with dextran surface extender

Ionic Capacity: 0.16-0.22 mmol Cl⁻/ml medium

pH stability, operational: 2-12

### Capto DEAE (Cytiva, Sweden):

Ligand: Diethylaminoethyl, partially quaternized amine groups

Particle size, d₅₀ᵥ: ˜90 μm

Matrix: Highly cross-linked agarose with dextran surface extender

Ionic Capacity: 0.29-0.35 mmol Cl⁻/ml medium

pH stability, operational: 2-9.

Further, the currently available Capto Q ImpRes (Cytiva, Sweden) was also tested as described below.

The support material of the ImpRes resin consists of substantially spherical particles or beads, which have a diameter of 40 μm.

### Equipment and Samples

Each resin was packed in a Tricorn 5 column (2 mL), 10 cm bead height and run using Agilent Bio-Inert 1260 system or an ÄKTA pure P25 system with a flow rate of 1 mL/min. Sample applied was approx. 1×10¹³ viral capsids of affinity purified AAV8 containing both full and empty capsids (>15% full capsids) for all the resins. Detection with fluorescence (excitation at 280 nm, emission 348 nm) or UV (280 and 260 nm).

### Results

Currently available cation resins (SP Sepharose XL, Capto SP ImpRes, Capto SP ImPact and Capto Adhere ImpRes; Cytiva, Sweden) were evaluated with 50 mM acetate pH 4.5 and 5.5 with a salt gradient up to 1M NaCl. Multimodal anion exchange resin (currently available Capto Adhere; Cytiva, Sweden) was evaluated with 20 mM Tris pH 6-9.5 and with a salt gradient up to 1M NaCl. The results for both types of resins tested showed no separation and only a single peak when applying affinity purified AAV8 (data not shown).

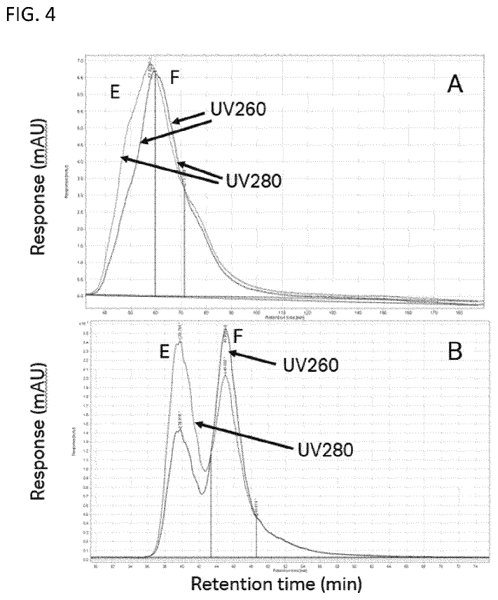

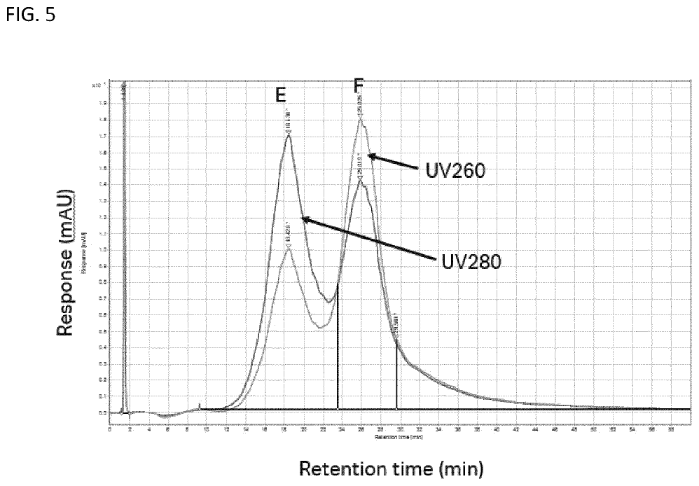

Each of the currently available resins Capto Q ImpRes (FIG. 4A), Capto Q (FIG. 4B) and Capto DEAE (FIG. 5) was run with 20 mM bis-Tris propane (BTP), pH 9.5 (for Capto Q ImpRes and Capto Q) or pH 7.5 (for Capto DEAE), 2 mM MgCl₂, 1% sucrose, 0.1% Poloxamer 188, with a linear gradient elution up to 400 mM of NaCl. Peak 1 area with UV 260:280 ratio below 1 was defined as the empty capsids and peak 2 area with UV 260:280 ratio above 1 was defined as full capsids. The % full capsids was calculated based on peak areas for UV260 and UV280 (Table 1). The peak content in terms of % full and empty capsids were also analyzed by qPCR (viral genome, full capsids) and ELISA (total capsids) and peak identity was confirmed (data not shown).

The chromatograms in FIG. 4 and FIG. 5 show that the dextran extender (present on Capto Q and Capto DEAE but not on Capto Q ImpRes) is dramatically improving the separation between full (F) and empty (E) AAV8 capsids. For Capto DEAE, reducing the pH to 7.5 improved the separation compared to higher pH up to 9.5 (data not shown).

The UV260:280 ratio was lower for Capto Q ImpRes (not including any dextran extender), indicating that the % full capsids is lower and thereby the enrichment is less efficient. For Capto Q and Capto DEAE (including a dextran extender), the ratio in peak 2 is higher showing that the % full capsids is higher and better separated from the empty capsids (Table 1). The calculated % full capsids based on area in peak 2 did not differ as much as the UV260:280 ratio, but the area determined for the full capsids is not accurate for Capto Q ImpRes (FIG. 4A) since the separation between full and empty capsids is poor. The full and empty peaks are overlapping and second half of peak is enriched for full capsids.

TABLE 1
Calculated % full capsids based on UV260:280 peak areas and
UV260:280 in peak 1 (empty capsids) and 2 (full capsids).

| | | |
|---|---|---|
| 260 nm | 280 nm | A260/A280 |

Capto Q ImpRes (pH 9.5)

| | | | |
|---|---|---|---|
| Peak 1 | 4196 | 5695 | 0.74 |
| Peak 2 | 3530 | 3283 | 1.08 |
| % full capsids (% peak 2 area) | 46%* | 36%* | |

Capto Q (pH 9.5)

| | | | |
|---|---|---|---|
| Peak 1 | 3467 | 5566 | 0.62 |
| Peak 2 | 4858 | 4062 | 1.20 |
| % full capsids (% peak 2 area) | 52% | 38% | |

Capto DEAE (pH 7.5)

| | | | |
|---|---|---|---|
| Peak 1 | 3340 | 5421 | 0.62 |
| Peak 2 | 4406 | 3657 | 1.20 |
| % full capsids (% peak 2 area) | 48% | 34% | |

*Overlapping peaks, inaccurate calculation of % full capsids.

### Example 2: Separation of AAV8 Capsids Under Variable Conditions

Experimental designs for separation of fully packaged AAV8 capsids from empty AAV8 capsids are performed with equipment and samples as in Example 1 above, and further by use of anion exchange chromatography material as in Example 1, with the following variations:

In terms of surface extenders:
1) Different size (kDa) of dextran (T10, T70, T250);
2) Different amounts of dextran;
3) Instead of dextran, use poly alcohol based on glycidol.

In terms of ligand density:
1) Capto Q: 60-160, 220-260 μmol/mL;
2) Capto DEAE: 150-290, 350-400 μmol/mL.

In terms of chromatography material support:
1) Smaller resin bead size: 35-90 μm;
2) Resin beads with larger pore sizes than Capto Q and Capto DEAE.

In terms of ligand chemistry:
1) Capto DEAE ligand with different levels of quaternization (ligand according to Formula III);
2) Capto Q analogues (wherein the Capto Q ligand is defined by Formula I):
   a. R1, R2 is ethyl; R3 is methyl;
   b. R1, R2 is methyl; R3 is CH2CHOHCH3;
3) Capto DEAE analogues (wherein the Capto DEAE ligand is defined by Formula II or III):
   a. m=1; n=1; R1, R2, R3, and R4=methyl; R5=H;
   b. m=1; n=1; R1, R2, R3, and R4=methyl; R5=CH2CHOHCH3;
   c. m=1; combination of two or more of the following structures (i)-(iv):
      (i) n is 0; R3 and R4 are methyl; R5 is hydrogen or CH2CHOHCH3;
      (ii) n is 1; R1, R2, R3, and R4 are methyl; R5 is hydrogen or CH2CHOHCH3;
      (iii) n is 2; each R1 and R2 is methyl; R3 and R4 is methyl; R5 is hydrogen or CH2CHOHCH3;
      (iv) n is 3; each R1 and R2 is methyl; R3 and R4 is methyl; R5 is hydrogen or CH2CHOHCH3.

In terms of buffers and elution conditions:
1) Different concentrations of MgCl₂ between 1-20 mM;
2) Different NaCl linear gradient 0.1-1M, with or without MgCl₂ as in 1);
3) Different pH linear gradient pH 4-10, with or without MgCl₂ as in 1);
4) Different buffers:
   a. Tris
   b. N-Methyldiethanolamine
5) Step elutions with pH, NaCl, MgCl₂ as in 1), 2) and 3);
6) Conditions suitable for flow-through separation, where one analyte binds to the chromatography material while another analyte does not bind to it (e.g., empty capsids bind while full capsids do not);
7) All of the above with or without additives like sucrose (0.1-5%) and poloxamer 188 detergent (0.01-1%).

### Example 3: Separation of Capsids of Different Adeno-Associated Virus Serotypes Under Variable Conditions

Experimental designs for separation of full capsids from empty capsids of adeno-associated virus serotypes AAV1, AAV2, AAV4, AAV5, AAV6, AAV7, AAV9, and AAV10, are performed according to the variable conditions of Example 1 and Example 2 above.

### Example 4: Separation of Capsids of Serotypes AAV2, AAV5, AAV8 and AAV9 On Anion Exchange Chromatography Materials Using Magnesium Chloride and Increasing Sodium Acetate Step Gradient

### Chromatography Materials

The same anion exchange chromatography materials as described in Example 1 (i.e., Capto Q, Capto DEAE and Capto Q ImpRes) were used for the separation of full and empty capsids of AAV2, AAV5, AAV8 and AAV9.

### Equipment and Samples

Each resin was packed in a Tricorn 5 column (2 mL) according to the packing instructions. The runs were performed using an Äkta Pure P25 system (P25-20031) with a flowrate of 1 CV/min (i.e., 2 mL/min), with the mixer of the system disconnected in order to minimize the dead volume and to get sharp conductivity steps. The sample was applied to the previously equilibrated column using a capillary loop. Typically, samples applied to each resin comprised affinity purified, or affinity and size exclusion purified, AAV2, AAV5, AAV8 or AAV9, respectively, at a concentration of approx. 5×10¹² AAV capsids, containing a mixture of full and empty capsids (>5% full capsids, as follows: AAV2 7-10%, AAV5 47%, AAV8 11-35%, AAV9 40%). The material needs to have low conductivity (1-3 mS/cm) to ensure binding of AAV to the anion exchange ligand.

The 280 and 260 nm UV absorbance were monitored during the runs and the 260/280 ratios were used as a diagnostic tool to navigate in the chromatogram and distinguish between full and empty capsid populations. The chromatograms were analyzed using the Evaluation package of Unicorn. A 260/280 ratio above 1.2 is considered to indicate 100% full capsids, and a 260/280 ratio of approx. or below 0.6-0.7 is considered to indicate 100% empty capsids. Blank runs with the buffers without AAV were performed to subtract any background signal if needed, to ensure removal of potential UV signals from the buffers.

### Process Conditions and Results

Currently available cation exchange resin, Capto S, and prototype cation exchange resin, Capto CM Dx ImpRes, were evaluated with acetate buffer at pH 4.5 and 5, with and without additives (0.1% poloxamer 188, 1% sucrose, with different elution salts (NaCl, NaOAc, NH₄Cl or NH₄SO₄ up to 500 mM) and additive salts (MgCl₂ and MgSO4 up to 20 mM), by applying an isocratic elution or continuous gradient elution, respectively. None of the above-mentioned conditions or resins resulted in a good baseline separation of full and empty capsids (data not shown).

Each of the currently available anion exchange resins with dextran extenders, Capto DEAE (strong, or partially strong, anion exchange) and Capto Q (strong anion exchange), were evaluated by applying a two-step elution method using a buffer system including a buffer A and a buffer B, both containing 20 mM Bis-Tris Propane (BTP) pH 9.0 and 2 mM MgCl₂, and buffer B additionally comprising 250 mM sodium acetate (NaOAc) as elution salt.

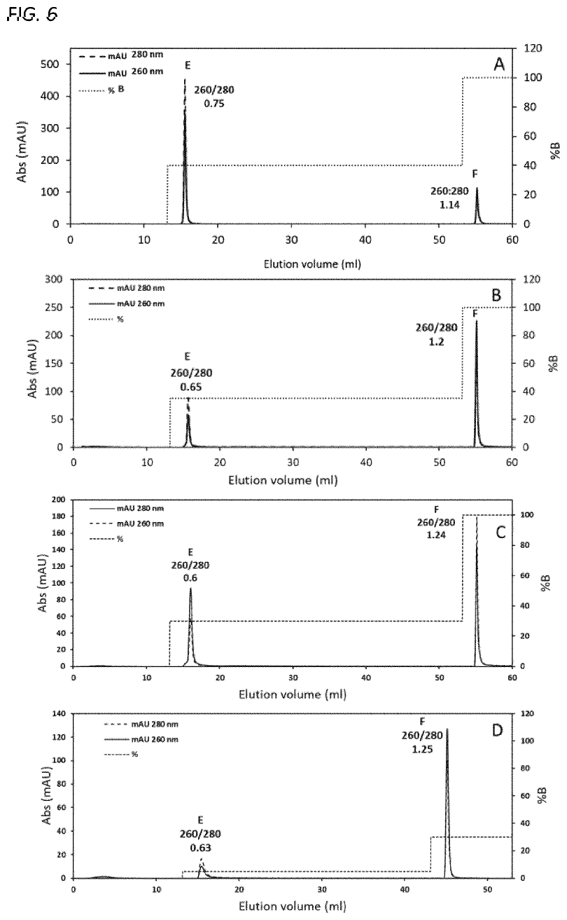

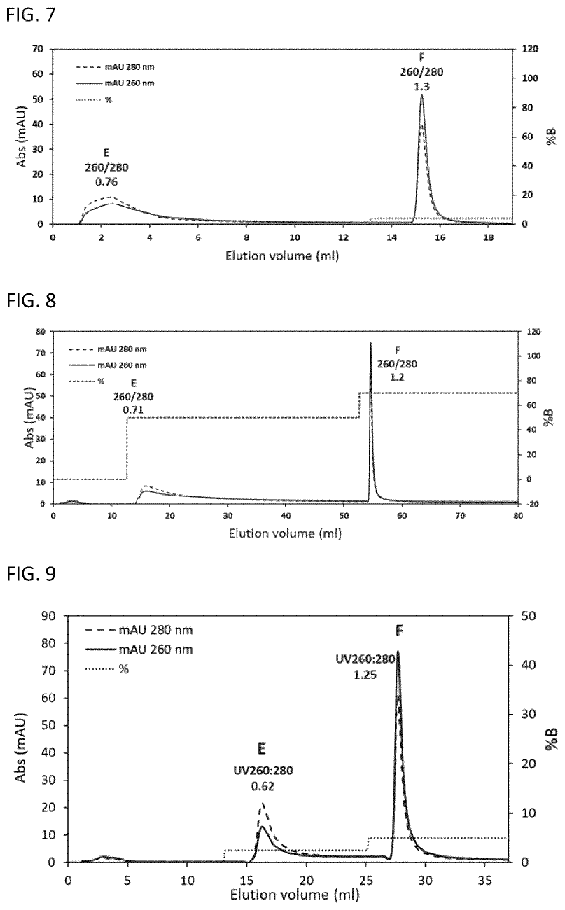

In FIGS. 6-7, the y-axis on the right-hand side of each chromatogram denotes the percentage of buffer B included in the resulting elution buffer (the rest being buffer A) during elution from the chromatography material.

Equilibration: 5 CV buffer A

Injection: empty loop/w 3 ml buffer A

Wash: 5 CV buffer A

Two-step elution: The conditions applied for each serotype are specified below

Re-equilibration: 5 CV buffer A

### Capto Q

AAV2: Step 1 40% B, 20 CV
Step 2 100% B, 5CV

AAV5: Step 1 35% B, 20 CV
Step 2 100% B, 5CV

AAV8: Step 1 30% B, 20 CV
Step 2 100% B, 5CV

AAV9: Step 1 5% B, 20 CV
Step 2 30% B, 5CV

When using the Capto Q resin, all tested serotypes (AAV2, AAV5, AAV8, and AAV9) resulted in a good separation of full and empty capsids (FIG. 6A-D). Elution of full (F) and empty (E) capsids, and UV260:280 ratios are indicated in FIG. 6A-D.

FIG. 6A shows the chromatogram for the NaOAc two-step elution of AAV2.

FIG. 6B shows the chromatogram for the NaOAc two-step elution of AAV5.

FIG. 6C shows the chromatogram for the NaOAc two-step elution of AAV8.

FIG. 6D shows the chromatogram for the NaOAc two-step elution of AAV9.

The % full capsids was calculated based on peak areas for UV260 and UV280. The peak content in terms of % full and empty capsids were also analyzed by qPCR (viral genome (VG), full capsids) and ELISA (total capsids). The results are summarized in Table 2.

TABLE 2
Results of the separation of full and empty capsids of four different serotypes applying a two-step
elution with increasing NaOAc concentration and constant magnesium chloride concentration.

| | | Peak 1 (empty capsids) | | | Peak 2 (full capsids) | | |
|---|---|---|---|---|---|---|---|
| | | UV | | | UV | | |
| | Start sample | 260:280 | VG | | 260:280 | VG | |
| | qPCR:ELISA | (peak | recovery | qPCR:ELISA | (peak | recovery | qPCR:ELISA |
| Serotype | (% full capsids) | area) | (%) | (% full capsids) | area) | (%) | (% full capsids) |
| AAV2 | 7-10% | 0.75 | NA | NA | 1.14 | NA | NA |
| AAV5 | 47% | 0.65 | 7 | 5 | 1.20 | 80 | 100 |
| AAV8 | 11-35% | 0.60 | 3 | 1 | 1.24 | 80 | 95 |
| AAV9 | 40% | 0.63 | 0.3 | 1 | 1.25 | 91 | 100 |

### Capto DEAE

When using the Capto DEAE resin, all tested AAV serotypes (AAV2, AAV5, AAV8, and AAV9) resulted in a good separation of full and empty capsids. However, they eluted at a lower percentage of buffer B compared to when being separated on the Capto Q resin.

FIG. 7 shows the results of the elution of AAV9 on the Capto DEAE resin. Using the same buffer system as described above for Capto Q (buffer A: 20 mM BTP pH 9.0, 2 mM MgCl₂; buffer B: buffer A+250 mM NaOAc), the AAV9 empty capsids eluted in flow-through and the AAV9 full capsids eluted when applying 4% of buffer B. The UV 260:280 ratio was 0.76 in the first peak (i.e., the flow-through peak), suggesting mainly empty AAV9 capsids but also a small amount of full capsids. The UV260:280 ratio was 1.3 in the second peak, indicating high purity of AAV9 full capsids (FIG. 7).

Capto Q ImpRes (without Dextran Extenders)

Capto Q ImpRes resin was evaluated for separation of AAV9 and AAV5, respectively, under conditions identical to those described above, except that a flowrate of 1 ml/min was applied due to high delta column pressures. The resin did not work for AAV5 but worked adequately for a two-step elution for separation of AAV9 full capsids from AAV9 empty capsids (results not shown). However, Capto Q ImpRes (without extenders) does not bind AAV9 empty capsids (which thereby elute in the flow-through) and only binds AAV9 full capsids weakly, and thus provides a less robust separation method than Capto Q (with extenders).

### Example 5: Separation of Capsids of Serotype AAV5 on Anion Exchange Chromatography Material Using Increasing Magnesium Chloride Step Gradient

The Capto Q resin was evaluated for separation of AAV5 by applying a two-step elution as described in Example 4, with the difference that buffer A and buffer B of the buffer system both included 20 mM Bis-Tris Propane (BTP) pH 7.0, 1% sucrose and 0.1% Pluronic, and buffer B additionally comprising 20 mM MgCl₂.

Equilibration: 5 CV buffer A

Injection: empty loop/w 3 ml buffer A

Wash: 5 CV buffer A

Step elution: step 1, 50% buffer B, 20 CV; step 2, 70% buffer B, 20 CV

Re-equilibration: 5 CV buffer A

FIG. 8 shows the results of the two-step elution method for AAV5 on Capto Q resin, applying 50% buffer B in the first step and 70% buffer B in the second step. Full (F) and empty (E) capsids, and UV260:280 ratios are indicated in FIG. 8.

### Example 6: Separation of Capsids of Serotype AAV5 on Anion Exchange Chromatography Material Using Magnesium Chloride and Increasing Sodium Chloride Step Gradient

The Capto Q resin was evaluated for separation of AAV5, by applying a two-step elution as described in Example 4 and Example 5, with the difference that buffer A and buffer B of the buffer system both included 20 mM Bis-Tris Propane (BTP) pH 9.5, 18 mM MgCl₂, 1% sucrose and 0.1% Pluronic, and buffer B additionally comprising 400 mM NaCl.

Equilibration: 5 CV buffer A

Injection: empty loop/w 3 ml buffer A

Wash: 5 CV buffer A

Two-step elution: step 1, 2.5% buffer B, 6 CV; step 2, 5% buffer B, 6 CV

Re-equilibration: 5 CV buffer A

FIG. 9 shows the results of the two-step elution method for AAV5 on Capto Q resin, applying 2.5% buffer B in the first step and 5% buffer B in the second step. Full (F) and empty (E) capsids, and UV260:280 ratios are indicated in FIG. 9. It is shown that the above-described conditions resulted in successful baseline separation of AAV5 capsids.

It is to be understood that the present disclosure is not restricted to the above-described exemplifying embodiments thereof and that several conceivable modifications of the present disclosure are possible within the scope of the following claims.

### REFERENCES

Weihong Qu et al, Scalable Downstream Strategies for Purification of Recombinant Adeno-Associated Virus Vectors in Light of the Properties, Current Pharmaceutical Biotechnology 2015 Aug.; 16(8): 684-695
Xiaotong Fu et al, Analytical Strategies for Quantification of Adeno-Associated Virus Empty Capsids to Support Process Development, Human gene therapy methods, 2019, 30(4): 144-152
Moelbert Susanne et al, Kosmotropes and chaotropes: modelling preferential exclusion, binding and aggregate stability, Biophysical Chemistry, 2004 Dec., 112(1): 45-57
Hyde Adam M et al, General Principles and Strategies for Salting-Out Informed by the Hofmeister Series, Organic Process Research & Development, 2017, 21 (9): 1355-1370.

1. A method for separating adeno-associated virus capsids fully packaged with genetic material from adeno-associated virus capsids not fully packaged with genetic material, the method comprising the following steps:
a. adding a liquid sample comprising adeno-associated virus capsids to a chromatography material,
wherein the liquid sample comprises adeno-associated virus capsids of a purity of at least 90% and of a concentration of at least 10¹² adeno-associated virus capsids/ml, of which at least 10% of the adeno-associated virus capsids are adeno-associated virus capsids fully packaged with genetic material,
wherein the chromatography material comprises a strong, or partially strong, anion exchange chromatography material comprising a support and a ligand for binding to the adeno-associated virus capsids,
wherein the chromatography material comprises a surface extender connecting the ligand to the support, wherein the surface extender is a polymer, wherein the polymer is selected from:
(i) a polymer having a naturally occurring skeleton, such as a polysaccharide, such as starch, cellulose, dextran, or agarose, and
(ii) a polymer having a synthetic skeleton, such as a polyvinyl alcohol, a polyacrylamide, a polymethacrylamide, or a polyvinyl ether;
b. eluting the adeno-associated virus capsids fully packaged with genetic material from the chromatography material;
wherein the adeno-associated virus capsids eluted in step (b) are eluted into eluate fractions, which eluate fractions combined comprise at least 50% of the adeno-associated virus capsids of the liquid sample added in step (a), of which at least 60% of the adeno-associated virus capsids are fully packaged with genetic material.

2. The method according to claim 1, wherein the ligand of the strong anion exchange chromatography material is defined by the following Formula I: 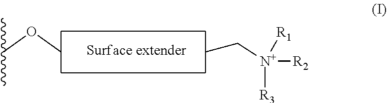
wherein
R₁ is selected from C1-C3 alkyl, and R₂ and R₃ are independently selected from C1-C3 alkyl, CH2OH, and CH2CHOHCH3.

3. The method according to claim 2, wherein each of R₁, R₂, and R₃ is CH3.

4. The method according to claim 1, wherein the ligand of the strong, or partially strong, anion exchange chromatography material is defined by the following Formula II: 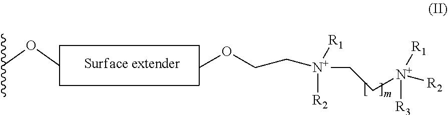
wherein:
m is an integer of from 1 to 3;
R₁ and R₂ are independently selected from a C1-C3 alkyl; R₃, and R₄ are independently selected from C1-C3 alkyl and CH2CHOHCH3; and R₅ is selected from hydrogen, a C1-C3 alkyl and CH2CHOHCH3;
provided that if m is 1, the ligand of the strong, or partially strong, anion exchange chromatography material is defined by the following Formula III: 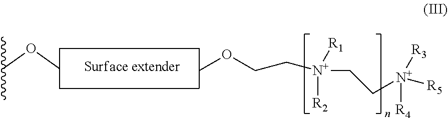
wherein n is an integer of from 0 to 3;
provided that if n is 0, R₃ and R₄ are independently selected from C1-C3 alkyl, and R₅ is hydrogen or CH2CHOHCH3.

5. The method according to claim 4, wherein the ligand is defined by Formula III and comprises a combination of two or more of the following structures (i)-(iv):
(i) n is 0; R₃ and R₄ are ethyl; and R₅ is hydrogen or CH2CHOHCH3;
(ii) n is 1; R₁, R₂, R₃, R₄ are ethyl; and R₅ is hydrogen or CH2CHOHCH3;
(iii) n is 2; each R₁ and R₂ is ethyl; R₃ and R₄ is ethyl; and R₅ is hydrogen or CH2CHOHCH3;
(iv) n is 3; each R₁ and R₂ is ethyl; R₃ and R₄ is ethyl; and R₅ is hydrogen or CH2CHOHCH3.

6. The method according to claim 1, wherein the surface extender is dextran.

7. The method according to claim 6, wherein the dextran has a molecular weight of from about 10 to about 2000 kDa, such as about 40 kDa.

8. The method according to claim 6, wherein the density of dextran is from about 5 to about 30 mg dextran per ml of the strong, or partially strong, anion exchange chromatography material.

9. The method according to claim 1, wherein steps (a) and (b) comprise applying a buffer having a pH of from about 6.0 to about 10.5, such as from about 7.5 to about 9.5, optionally wherein said buffer comprises tris(hydroxymethyl)amino-methane (i.e., Tris), 1,3-bis(tris(hydroxymethyl)methylamino) propane (i.e., bis-Tris propane), triethanolamine, N-methyldiethanolamine, Diethanolamine, 1,3-diaminopropane, or ethanolamine.

10. The method according to claim 1, wherein step (b) comprises applying a buffer comprising a compound which improves separation between capsids fully packaged with genetic material and capsids not fully packaged with genetic material, optionally wherein said compound is selected from a carbohydrate, a divalent metal ion, and a detergent;
optionally wherein the carbohydrate is selected from sucrose, sorbitol, and a polysaccharide;
optionally wherein the divalent metal ion is selected from Mg²⁺, Fe²⁺, and Mn²⁺, optionally wherein the divalent metal ion is present in the form of a salt, optionally in combination with chloride ions or sulphate ions;
optionally wherein the detergent is selected from poloxamer and polysorbate.

11. The method according to claim 1, wherein the support of the chromatography material comprises particles, nanofibres, a monolith, or a membranous structure;
optionally wherein the particles are particles having a homogeneous porosity and being at least partly permeable to adeno-associated virus capsids;
optionally wherein the particles are substantially spherical particles;
optionally wherein the nanofibres comprise electrospun polymer nanofibres;
optionally wherein the membranous structure comprises a nonwoven web of polymer nanofibers.

12. The method according to claim 1, wherein the chromatography material is a polishing chromatography material.

13. The method according to claim 1, wherein the liquid sample added in step (a), is a pre-purified liquid sample.

14. The method according to claim 1, further comprising (a1) pre-purifying adeno-associated virus capsids by separating adeno-associated virus capsids from an adeno-associated virus capsid-containing cell culture harvest, thereby obtaining a pre-purified liquid sample comprising adeno-associated virus capsids, before adding said pre-purified liquid sample comprising adeno-associated virus capsids to the chromatography material according to step (a), optionally wherein said pre-purifying comprises subjecting the adeno-associated virus capsid-containing cell culture harvest to chromatography, or to clarification followed by chromatography.

15. The method according to claim 1, further comprising subjecting the eluate fractions comprising adeno-associated virus capsids fully packaged with genetic material, eluted in step (b), to one or more of the following steps:
c1) concentrating the adeno-associated virus capsids to a pharmaceutically relevant dose, c2) replacing a buffer applied in step (b), with a pharmaceutically acceptable buffer, and/or
c3) sterilizing the eluate fractions comprising adeno-associated virus capsids, thereby obtaining a pharmaceutical composition comprising adeno-associated virus capsids.

16. The method according to claim 1, wherein the adeno-associated virus capsids are capsids of adeno-associated virus serotype 8 (AAV8), adeno-associated virus serotype 5 (AAV5), adeno-associated virus serotype 1 (AAV1), adeno-associated virus serotype 2 (AAV2), adeno-associated virus serotype 4 (AAV4), adeno-associated virus serotype 6 (AAV6), adeno-associated virus serotype 7 (AAV7), adeno-associated virus serotype 9 (AAV9), or adeno-associated virus serotype 10 (AAV10), or a variant thereof.

17. The method according to claim 1, wherein the elution buffer of step (b) comprises a kosmotropic salt, wherein the salt comprises (i) an anion selected from a group consisting of CO₃²⁻, SO₄²⁻, S₂O₃²⁻, H₂PO₄⁻, HPO₄²⁻, acetate⁻, citrate⁻, and Cl⁻, and (ii) a cation selected from a group consisting of NH₄⁺, K⁺, Na⁺, and Li⁺; optionally wherein the salt is sodium acetate.

18. The method according to claim 1, wherein the chromatography material is defined by Formula IV: 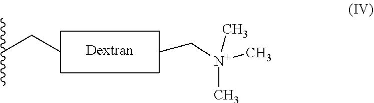
and wherein the elution buffer of step (b) comprises sodium acetate,
optionally wherein the adeno-associated virus capsids are capsids of adeno-associated virus serotype 9 (AAV9) or a variant thereof.

19. A method for preventing or treating a disease or disorder related to an organ or tissue in a subject, optionally by gene therapy, comprising administering to the subject a pharmaceutical composition comprising adeno-associated virus capsids obtained by performing the method of claim 15, in which pharmaceutical composition the ratio of adeno-associated virus capsids fully packaged with genetic material to adeno-associated virus capsids not fully packaged with genetic material is at least 3:2, preferably at least 4:1;
optionally wherein
(i) the capsids are selected from AAV1, AAV2, AAV4, AAV5, AAV8, and AAV10 capsids, or a variant thereof, and the organ or tissue is the central nervous system;
(ii) the capsids are selected from AAV1, and AAV8 capsids, or a variant thereof, and the organ or tissue is the heart;
(iii) the capsids are AAV2 capsids or a variant thereof, and the organ or tissue is kidney;
(iv) the capsids are selected from AAV7, and AAV8 capsids, or a variant thereof, and the organ or tissue is liver;
(v) the capsids are selected from AAV4, AAV5, and AAV6 capsids, or a variant thereof, and the organ or tissue is lung;
(vi) the capsids are AAV8 or a variant thereof, and the organ or tissue is pancreas;
(vii) the capsids are selected from AAV2, AAV5, and AAV8 capsids, or a variant thereof, and the organ or tissue is photoreceptor cells;
(viii) the capsids are selected from AAV1, AAV2, AAV4, AAV5, and AAV8 capsids, or a variant thereof, and the organ or tissue is retinal pigment epithelium;
(ix) the capsids are selected from AAV1, AAV6, AAV7, and AAV8 capsids, or a variant thereof, and the organ or tissue is skeletal muscle;
(x) the capsids are AAV10 capsids or a variant thereof, and the organ or tissue is brain; or
(xi) the capsids are AAV9 capsids or a variant thereof, and the organ or tissue is selected from the central nervous system, heart, liver, lung, and skeletal muscle.

20. The method according to claim 1, wherein the not fully packaged adeno-associated virus capsids are empty adeno-associated virus capsids and/or partially packaged adeno-associated virus capsids.

21. A composition comprising adeno-associated virus capsids obtained by performing the method of claim 1, in which composition the ratio of adeno-associated virus capsids fully packaged with genetic material to adeno-associated virus capsids not fully packaged with genetic material is at least 3:2, preferably at least 4:1.

22. A pharmaceutical composition comprising adeno-associated virus capsids obtained by performing the method of claim 15, in which pharmaceutical composition the ratio of adeno-associated virus capsids fully packaged with genetic material to adeno-associated virus capsids not fully packaged with genetic material is at least 3:2, preferably 4:1.

23. The pharmaceutical composition according to claim 1 for use in therapy, optionally for use in gene therapy.

24. The pharmaceutical composition for use according to claim 1, wherein the adeno-associated virus capsids are capsids of adeno-associated virus serotype 8 (AAV8), adeno-associated virus serotype 5 (AAV5), adeno-associated virus serotype 1 (AAV1), adeno-associated virus serotype 2 (AAV2), adeno-associated virus serotype 4 (AAV4), adeno-associated virus serotype 6 (AAV6), adeno-associated virus serotype 7 (AAV7), adeno-associated virus serotype 9 (AAV9), or adeno-associated virus serotype 10 (AAV10), or a variant thereof,
and wherein the composition is for use in gene therapy;
optionally wherein the pharmaceutical composition is for use in the prevention or treatment of a disease or a disorder related to an organ or tissue, wherein
(i) the capsids are selected from AAV1, AAV2, AAV4, AAV5, AAV8, and AAV10 capsids, and the organ or tissue is the central nervous system;
(ii) the capsids are selected from AAV1, and AAV8 capsids, and the organ or tissue is the heart;
(iii) the capsids are AAV2 capsids, and the organ or tissue is kidney;
(iv) the capsids are selected from AAV7, and AAV8 capsids, and the organ or tissue is liver;
(v) the capsids are selected from AAV4, AAV5, and AAV6 capsids, and the organ or tissue is lung;
(vi) the capsids are AAV8, and the organ or tissue is pancreas;
(vii) the capsids are selected from AAV2, AAV5, and AAV8 capsids, and the organ or tissue is photoreceptor cells;
(viii) the capsids are selected from AAV1, AAV2, AAV4, AAV5, and AAV8 capsids, and the organ or tissue is retinal pigment epithelium;
(ix) the capsids are selected from AAV1, AAV6, AAV7, and AAV8 capsids, and the organ or tissue is skeletal muscle;
(x) the capsids are AAV10 capsids and the organ or tissue is brain; or
(xi) the capsids are AAV9 capsids or a variant thereof, and the organ or tissue is selected from the central nervous system, heart, liver, lung, and skeletal muscle.

25. The composition according to claim 21, wherein the not fully packaged adeno-associated virus capsids are empty adeno-associated virus capsids and/or partially packaged adeno-associated virus capsids.

26. Use of an anion exchange chromatography material comprising a support, a ligand, and a surface extender connecting the ligand to the support, and being defined by Formula IV: 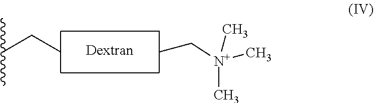
for separating adeno-associated virus capsids fully packaged with genetic material from adeno-associated virus capsids not fully packaged with genetic material, comprising performing the following steps:
a. adding a liquid sample comprising adeno-associated virus capsids of a purity of at least 90% and of a concentration of at least 10¹² adeno-associated virus capsids/ml, of which at least 10% of the adeno-associated virus capsids are adeno-associated virus capsids fully packaged with genetic material, to the chromatography material;
b. eluting the adeno-associated virus capsids fully packaged with genetic material from the chromatography material;
wherein the adeno-associated virus capsids eluted in step (b) are eluted into eluate fractions, which eluate fractions combined comprise at least 50% of the adeno-associated virus capsids of the liquid sample added in step (a), of which at least 60% of the adeno-associated virus capsids are fully packaged with genetic material.

27. The use of claim 26, wherein the elution buffer of step (b) comprises sodium acetate;
wherein the adeno-associated virus capsids are capsids of adeno-associated virus serotype 1 (AAV1), adeno-associated virus serotype 2 (AAV2), adeno-associated virus serotype 4 (AAV4), adeno-associated virus serotype 5 (AAV5), adeno-associated virus serotype 6 (AAV6), adeno-associated virus serotype 7 (AAV7), adeno-associated virus serotype 8 (AAV8), adeno-associated virus serotype 9 (AAV9), or adeno-associated virus serotype 10 (AAV10), or a variant thereof;
optionally wherein the adeno-associated virus capsids are capsids of adeno-associated virus serotype 9 (AAV9) or a variant thereof.
